(19) United States
(12) Patent Application Publication
(10) Pub. No.: US 2026/0216487 A1
(43) Pub. Date: Jul. 30, 2026

(54) INGESTIBLE DEVICE WITH MULTI-STAGE ACTUATOR

(71) Applicant: Rani Therapeutics, LLC, San Jose, CA (US)

(72) Inventors: Mir IMRAN, Los Altos Hills, CA (US); Baber SYED, San Jose, CA (US); Farzad FOROUZANDEH, San Jose, CA (US)

(73) Assignee: Rani Therapeutics, LLC, San Jose, CA (US)

(21) Appl. No.: 19/143,588

(22) PCT Filed: Dec. 26, 2023
(86) PCT No.: PCT/US2023/085883

§ 371 Date: Jun. 26, 2025

Related U.S. Application Data
(60) Provisional application No. 63/435,526, filed on Dec. 27, 2022.

(51) Int. Cl. A61M 31/00; A61M 5/14; A61M 5/142; A61M 5/145; A61M 37/00

(52) U.S. Cl. CPC A61M 31/002; A61M 5/1408; A61M 5/14526; A61M 37/0069; A61M 2005/14284; A61M 2210/1017; A61M 2210/106; A61M 2230/208

(57) ABSTRACT

An ingestible device (200) for delivering a therapeutic preparation into a Gl lumen wall or surrounding tissue of a subject includes an expandable component (206) and a delivery system (300). The expandable component includes a first actuation mechanism (215,216,218). The delivery system is coupled to the expandable component and includes a second actuation mechanism (305) and the therapeutic preparation (306). The therapeutic preparation includes at least one therapeutic agent. The first actuation mechanism is structured to cause the expandable component to expand so as to position the delivery system relative to a surface of the Gl lumen wall. The second actuation mechanism is structured to generate a second force within the delivery system. The delivery system is structured to deliver the therapeutic preparation into the Gl lumen wall or surrounding tissue.

### CROSS-REFERENCE TO RELATED APPLICATION

This application claims the benefit of and priority to U.S. Provisional Application No. 63/435,526, filed on Dec. 27, 2022, the entire contents of which are hereby incorporated by reference.

### BACKGROUND

Ingestible devices have been proposed to orally deliver therapeutic preparations including a therapeutic agent into a gastrointestinal (GI) lumen wall of a subject for systemic uptake of the therapeutic agent. Some ingestible devices include an actuation mechanism that can generate a sufficient actuation force to deliver the therapeutic preparation from the device into the GI lumen wall such that the therapeutic agent reaches the blood stream.

### SUMMARY

Embodiments of the present disclosure relate generally to ingestible devices, systems, and methods for delivering a therapeutic preparation into a GI lumen wall or surrounding tissue (e.g., a peritoneum or peritoneal cavity) of a subject for treating a disease or condition.

In one aspect, an ingestible device for delivering a therapeutic preparation into a GI lumen wall or surrounding tissue of a subject includes an expandable component and a delivery system. The expandable component includes a first actuation mechanism. The delivery system is coupled to the expandable component and includes a second actuation mechanism and the therapeutic preparation. The therapeutic preparation includes at least one therapeutic agent. The first actuation mechanism is structured to generate a first force in response to a condition in the GI tract of the subject to cause the expandable component to expand so as to position the delivery system relative to a surface of the GI lumen wall. The second actuation mechanism is structured to generate a second force within the delivery system in response to the first force reaching a first threshold value. The delivery system is structured to deliver the therapeutic preparation into the GI lumen wall or surrounding tissue upon the second force reaching a second threshold value.

In another aspect, provided herein are methods of delivering a therapeutic preparation into a GI lumen wall or surrounding tissue of a subject via an ingestible device as described herein.

In one or embodiments of any of the foregoing aspects, the first threshold value is associated with an expanded state of the expandable component within the GI tract.

In one or embodiments of any of the foregoing aspects, the second threshold value is greater than the first threshold value.

In one or embodiments of any of the foregoing aspects, the first actuation mechanism includes a first gas generating mechanism structured to generate a first gas pressure to provide the first force. In some embodiments, the first gas generating mechanism includes a first plurality of substances that when mixed undergo a first chemical reaction to generate the first gas pressure. In some embodiments, the first chemical reaction is initiated in response to the condition in the GI tract of the subject. In some embodiments, the condition is a pH in the small intestine. In some embodiments, the first gas pressure is in a range of about 5-20 psi.

In one or embodiments of any of the foregoing aspects, the second actuation mechanism includes a second gas generating mechanism structured to generate a second gas pressure to provide the second force. In some embodiments, the second gas generating mechanism includes a second plurality of substances that when mixed undergo a second chemical reaction to generate the second gas pressure. In some embodiments, the second gas pressure is in a range of about 25-250 psi.

In one or embodiments of any of the foregoing aspects, the second threshold value corresponds with a desired penetration depth in the subject for delivery of the therapeutic preparation. In some embodiments, the desired penetration depth is into a peritoneum or a peritoneal cavity of the subject. In some embodiments, the desired penetration depth is into a submucosa of the GI lumen wall.

In one or embodiments of any of the foregoing aspects, the second force is substantially contained within an interior of the delivery system.

In one or embodiments of any of the foregoing aspects, the first force is substantially contained within the expandable component external to the delivery system.

In one or embodiments of any of the foregoing aspects, the therapeutic preparation is a first therapeutic preparation and the delivery system further includes a second therapeutic preparation. In some embodiments, the first therapeutic preparation includes the at least one therapeutic agent, and the second therapeutic preparation includes a second therapeutic agent. In some embodiments, the at least one therapeutic agent is the same as, or different from, the second therapeutic agent.

In one or embodiments of any of the foregoing aspects, the delivery system is a first delivery system and the therapeutic preparation is a first therapeutic preparation, wherein the device further includes a second delivery system coupled to the expandable component, and wherein the second delivery system includes a second therapeutic preparation.

In one or embodiments of any of the foregoing aspects, the delivery system includes a housing and a delivery component, wherein the housing includes a first chamber and a second chamber, and wherein the delivery component is movably disposed in the housing. In some embodiments, the first chamber is oriented perpendicularly relative to the second chamber. In some embodiments, the second actuation mechanism is disposed in the first chamber, and the therapeutic preparation is disposed in the second chamber. In some embodiments, the second actuation mechanism includes a first substance, a second substance, and a separator, wherein the first and second substances are disposed in the first chamber and are separated from each other by the separator. In some embodiments, the device is structured such that the first force causes the first and second substances to mix to generate a gas pressure to provide the second force within the first chamber, and wherein the delivery component is structured to deliver the therapeutic preparation from the second chamber in response to the generated gas pressure. In some embodiments, the separator includes a membrane. In some embodiments, the membrane is structured to open in response to a pressure applied to the membrane. In some embodiments, the membrane is a pierceable membrane. In some embodiments, the delivery system further includes an actuation piston structured to pierce the pierceable membrane in response to the first force to cause the first and second substances to mix. In some embodiments, the separator includes a substrate with an opening disposed therein and a plug detachably coupled to the substrate at the opening. In some embodiments, the delivery system further includes a compressible bladder structured to compress in response to the first force to detach the plug from the substrate to cause the first and second substances to mix. In some embodiments, the delivery system further includes a valve structured to open in response to the first force to cause the first and second substances to mix. In some embodiments, the therapeutic preparation is delivered in a fluid form from the second chamber. In some embodiments, the housing includes a nozzle to deliver the therapeutic preparation as a fluid jet from the second chamber into the GI lumen wall or surrounding tissue thereof. In some embodiments, the delivery system further includes a hollow needle coupled to the delivery component, and wherein the needle is structured to penetrate the GI lumen wall or surrounding tissue to deliver the fluid form of the therapeutic preparation. In some embodiments, the therapeutic preparation includes a biodegradable needle including a cavity and a solid tablet comprising the therapeutic agent, and wherein the solid tablet is disposed in the cavity. In some embodiments, the therapeutic preparation is formed into the shape of a needle.

In one or embodiments of any of the foregoing aspects, the expandable component includes a balloon. In some embodiments, the balloon comprises a biodegradable material such that at least a portion of the balloon degrades within the GI tract of the subject upon delivery of the therapeutic preparation.

In another aspect, a delivery system for an ingestible device includes a first housing, a first piston, a needle, a drug container, and a fluid preparation. The first housing includes a piston chamber defining a longitudinal axis. The first piston is disposed in the piston chamber. The needle is coupled to the first piston. The drug container is coupled to the first housing and is in selective fluid communication with the piston chamber. The drug container is oriented laterally relative to the first housing. The fluid preparation is disposed in the drug container. The drug container includes a flexible material structured to deform upon ingestion of the device to expel the fluid preparation into the piston chamber. In some embodiments, the delivery system further includes a second housing surrounding at least a portion of the drug container and a second piston disposed in the second housing adjacent to the drug container. In some embodiments, the first piston is structured to move in a longitudinal direction within the first housing to cause the needle to penetrate a GI lumen wall, and the second piston is structured to move in a lateral direction within the second housing to deform the drug container to expel the fluid preparation. In some embodiments, the second housing includes a vent channel to vent gas to a GI lumen environment.

In another aspect, an ingestible device includes an expandable component and a delivery system as described herein. The expandable component includes a first actuation mechanism.

In another aspect, an ingestible device for delivering a therapeutic preparation into a GI lumen wall or surrounding tissue of a subject includes an expandable component, a modular delivery system coupled to the expandable component, and a therapeutic preparation disposed in the modular delivery system. The therapeutic preparation includes at least one therapeutic agent. The expandable component includes a first gas generating mechanism structured to generate a first gas pressure within the expandable component. The modular delivery system includes a second gas generating mechanism structured to generate a second gas pressure within the modular delivery system in response to the first gas pressure. The expandable component is structured to expand in response to the first gas pressure to position the delivery system relative to a surface of the GI lumen wall. The modular delivery system is structured to deliver the therapeutic preparation into the GI lumen wall or surrounding tissue in response to the second gas pressure.

In another aspect, an ingestible device for delivering a therapeutic preparation into a GI lumen wall or surrounding tissue of a subject includes an expandable component, a modular delivery system coupled to the expandable component, and a therapeutic preparation disposed in the modular delivery system. The therapeutic preparation includes at least one therapeutic agent. The expandable component includes a first actuation mechanism structured to generate a first force within the expandable component. The modular delivery system includes a second actuation mechanism structured to generate a second force within the modular delivery system in response to the first force reaching a first threshold value. The expandable component is structured to expand to position the delivery system relative to a surface of the GI lumen wall. The modular delivery system is structured to deliver the therapeutic preparation into the GI lumen wall or surrounding tissue in response to the second force reaching a second threshold value.

In another aspect, a modular delivery system for an ingestible device to deliver a therapeutic preparation into a GI lumen wall or surrounding tissue of a subject includes a housing, a gas generating mechanism, a delivery component, and the therapeutic preparation comprising at least one therapeutic agent. The housing defines a first chamber and a second chamber. The delivery component is movably disposed in the housing between the first chamber and the second chamber. The gas generating mechanism is disposed in the first chamber. The therapeutic preparation is disposed in the second chamber. The gas generating mechanism is structured to generate a gas pressure within the first chamber in response to a force applied to the modular delivery system. The delivery component is structured to deliver the therapeutic preparation from the second chamber into the GI lumen wall or surrounding tissue in response to the gas pressure generated within the first chamber.

In another aspect, a modular delivery system for an ingestible device to deliver a therapeutic preparation into a GI lumen wall or surrounding tissue of a subject includes a housing, a first substance, a second substance, a separator, a delivery piston movably disposed in the housing, and the therapeutic preparation comprising at least one therapeutic agent. The first and second substances are disposed in the housing and are separated from each other by the separator. The modular delivery system is structured such that the first and second substances mix to form a gas in response to a force applied to the modular delivery system. The gas creates a pressure within the housing to cause the delivery piston to deliver the therapeutic preparation from the housing into the GI lumen wall or surrounding tissue.

In another aspect, a method for delivering a therapeutic preparation into a GI lumen wall or surrounding tissue of a subject includes ingesting an ingestible device, where the device includes an expandable component and a modular delivery system coupled to the expandable component. The therapeutic preparation comprises at least one therapeutic agent and is disposed in the modular delivery system. The expandable component comprises a first gas generating mechanism. The modular delivery system comprises a second gas generating mechanism. Upon the device reaching a desired location in the GI tract, the first gas generating mechanism generates a first gas pressure within an interior of the expandable component to cause the expandable component to expand to position the delivery system relative to a surface of the GI lumen wall. The second gas generating mechanism generates a second gas pressure within the modular delivery system in response to the first gas pressure to deliver the therapeutic preparation into the GI lumen wall or surrounding tissue.

In another aspect, a method for delivering a therapeutic preparation into a GI lumen wall or surrounding tissue of a subject includes ingesting an ingestible device, where the device includes an expandable component and a modular delivery system coupled to the expandable component. The therapeutic preparation comprises at least one therapeutic agent and is disposed in the modular delivery system. The expandable component comprises a first actuation mechanism disposed in the expandable component external to the modular delivery system. The modular delivery system comprises a second actuation mechanism disposed within the modular delivery system. Upon the device reaching a desired location in the GI tract, the first actuation mechanism generates a first force within an interior of the expandable component. Upon the first force reaching a first threshold value, the second actuation mechanism generates a second force within the modular delivery system. Upon the second force reaching a second threshold value, the modular delivery system is structured to deliver the therapeutic preparation into the GI lumen wall or surrounding tissue.

The foregoing general description and following detailed description are provided by way of example and are intended to provide further explanation of the disclosure, without being limiting. Other objects, advantages, and novel features will be readily apparent to those skilled in the art from the following brief description of the drawings and detailed description of the disclosure.

### BRIEF DESCRIPTION OF THE DRAWINGS

FIG. 1 illustrates an embodiment of an ingestible device in block diagrammatic form.

FIG. 2 illustrates a cross-sectional view of an embodiment of the device of FIG. 1.

FIG. 3 illustrates a detail view of a portion of the device of FIG. 2.

FIG. 4 illustrates the device of FIG. 3 in a second stage when the device has reached a desired location in a GI tract of a subject to deliver a therapeutic preparation.

FIGS. 5-8 illustrate various embodiments of a modular delivery system of the ingestible device of FIG. 1.

FIG. 9 illustrates a method of delivering a therapeutic preparation into a GI lumen wall or surrounding tissue of a subject using the devices and systems of the present disclosure.

FIG. 10 illustrates a cross-sectional view of another embodiment of a delivery system of the ingestible device of FIG. 1.

### DETAILED DESCRIPTION

Before discussing details of the devices, assemblies, and methods of the present disclosure, a few conventions are provided for the convenience of the reader.

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural references unless the context clearly dictates otherwise. Reference to an object in the singular is not intended to mean “one and only one” unless explicitly so stated, but rather “one or more.”

As used herein, a phrase in the form “A/B” or in the form “A and/or B” means (A), (B), or (A and B); a phrase in the form “at least one of A, B, or C” means (A), (B), (C), (A and B), (A and C), (B and C), or (A, B, and C).

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be omitted or can be used in another embodiment (e.g., in another embodiment illustrated and described herein, or in another embodiment within the scope of the present disclosure and not illustrated and/or not described herein).

The term “component” refers herein to one item of a set of one or more items that together make up a device, a composition, or a system under discussion. A component may be in a solid, powder, gel, plasma, fluid, gas, or other constitution. For example, a device may include multiple solid components which are assembled together to structure the device and may further include a fluid component that is disposed in the device. For another example, a composition may include a single component, or two or more components which are mixed together to make the composition. A composition may be in the form of a fluid, a slurry, a powder, or a solid (e.g., in a condensed or a consolidated form such as a tablet or microtablet). A device or system can include one or more compositions and/or one or more other components.

The term “design” or a grammatical variation thereof (e.g., “designing” or “designed”) refers herein to characteristics intentionally incorporated based on, for example, estimates of tolerances (e.g., component tolerances and/or manufacturing tolerances) and estimates of environmental conditions expected to be encountered (e.g., temperature, humidity, external or internal ambient pressure, external or internal mechanical pressure, stress from external or internal mechanical pressure, age of product, or shelf life, or, if introduced into a body, physiology, body chemistry, biological composition of fluids or tissue, chemical composition of fluids or tissue, pH, species, diet, health, gender, age, ancestry, disease, or tissue damage); it is to be understood that actual tolerances and environmental conditions before and/or after delivery can affect characteristics so that different components, devices, compositions, or systems with a same design can have different actual values with respect to those characteristics. Design encompasses also variations or modifications before or after manufacture.

The term “structured” or a grammatical variation thereof (e.g., “structure” or “structuring”) refers herein to a component, device, composition, or system that is manufactured according to a concept or design or variations thereof or modifications thereto (whether such variations or modifications occur before, during, or after manufacture) whether or not such concept or design is captured in a writing.

The term “body” refers herein to an animalia body.

The term “subject” refers herein to a body into which an embodiment of the present disclosure is, or is intended to be, delivered. For example, with respect to humans, a subject may be a patient under treatment of a health care professional. The terms “individual,” “subject,” and “patient” may be used interchangeably herein, and refer to any individual animalia subject (e.g., bovine, canine, feline, equine, or human). In specific embodiments, the subject, individual, or patient is a human.

The term “fluid” refers herein to a liquid or gas, and encompasses moisture and humidity. The term “fluidic environment” refers herein to an environment in which one or more fluids are present.

The term “ingest” or a grammatical variation thereof (e.g., “ingesting”, “ingestion,” or “ingested”) refers herein to taking into the stomach, whether by swallowing or by other means of depositing into the stomach (e.g., by depositing into the stomach by endoscope or depositing into the stomach via a port).

The term “degrade” or a grammatical variation thereof (e.g., “degrading”, “degraded”, “degradable”, and “degradation”) refers herein to weakening, partially degrading, or fully degrading, such as by dissolution, chemical degradation (including biodegradation), decomposition, chemical modification, mechanical degradation, or disintegration, which encompasses also, without limitation, dissolving, crumbling, deforming, shriveling, or shrinking. The term “non-degradable” refers to an expectation that degradation will be minimal, or within a certain acceptable design percentage, for at least an expected duration in an expected environment.

The term “degradation rate” or a grammatical variation thereof (e.g., “rate of degradation”) refers herein to a rate at which a material degrades. A designed degradation rate of a material in a particular implementation can be defined by a rate at which the material is expected to degrade under expected conditions (e.g., in physiological conditions) at a target delivery site. A designed degradation time for a particular implementation can refer to a designed time to complete degradation or a designed time to a partial degradation sufficient to accomplish a design purpose (e.g., breach). Accordingly, for example, a designed degradation time can be specific to a component and/or specific to expected conditions at a target delivery site. A designed degradation time can be short or long and can be defined in terms of approximate times, maximum times, or minimum times.

The terms “substantially”, “about”, and similar terms are used herein to describe and account for small variations which may result from, for example, a manufacturing or assembly process. For example, when used in conjunction with a numerical value, the terms can refer to a variation in the value of less than or equal to ±10%.

The term “lumen” refers herein to the inside space of a tubular structure. Examples of lumens in a body include arteries, veins, and tubular cavities within organs.

The term “lumen wall” refers to a wall of a lumen, where the wall includes all layers from an inner perimeter to an outer perimeter of the lumen, such as, with respect to lumens in a body, the mucosa, submucosa, muscularis, serosa, and an outer wall of the lumen, with the constituent blood vessels and tissues.

The term “gastrointestinal tract” or “GI tract” refers herein to the intake/expulsion system of a body including, for example, the mouth, pharynx, esophagus, stomach, pylorus, small intestine, cecum, large intestine, colon, rectum, anus, and valves or sphincters therebetween.

The term “GI lumen” refers generally to any lumen of the GI tract (e.g., a lumen of the esophagus, stomach, small intestine, large intestine, or colon) and the term “GI lumen wall” refers to a lumen wall of a GI lumen.

As used herein, the terms “comprising”, “comprise”, “comprises”, “includes”, and “including” are intended to mean that the compositions and methods include the recited elements, but do not exclude others.

Referring generally to the Figures, disclosed herein are embodiments relating to devices, systems, and methods for delivering a therapeutic preparation into a GI lumen wall or surrounding tissue (e.g., a peritoneum or peritoneal cavity) of a GI tract of a subject to treat a disease or condition. The disclosed devices are advantageously structured with a multi-stage actuation mechanism. More specifically, the disclosed devices include a delivery system that includes a self-contained actuation mechanism. The self-contained actuation mechanism can generate a localized force (e.g., localized pressure, localized mechanical force) within the delivery system in response to an external force applied to the delivery system to thereby deliver one or more therapeutic preparations into the GI lumen wall or surrounding tissue thereof. The disclosed devices further include an expandable component to locate and/or to align the delivery system relative to a surface of the GI lumen wall at a desired location in the GI tract of the subject. Expansion of the expandable component can provide the external force to initiate actuation of the self-contained actuation mechanism in a sequential, multi-stage manner.

In this way, the disclosed devices and systems can generate sufficiently high actuation forces to deliver, for example, a variety of different dosages, different dosage forms (e.g., solid and liquid therapeutic preparation), and multiple therapeutic preparations. In addition, as discussed herein, the multi-stage design can allow for flexibility to use different actuation mechanisms and materials (e.g., dissolvable materials) for various components of the device. Further, the multi-stage design may help to optimize the device package size and device shelf life.

FIG. 1 illustrates in block diagrammatic form an example of an ingestible device 100 according to one or more embodiments of the present disclosure. Device 100 includes an expandable component 102, a delivery system 104 coupled to expandable component 102, and at least one therapeutic preparation 106 disposed in delivery system 104. Expandable component 102 includes a first actuation mechanism 103 and delivery system 104 includes a second actuation mechanism 105. First actuation mechanism 103 is structured to generate a first force (e.g., a pressure, a mechanical force) within expandable component 102 external to delivery system 104. The first force may cause expandable component 102 to expand. In response to the first force, second actuation mechanism 105 is structured to generate a second, local force (e.g., a local pressure, a local mechanical force) within delivery system 104 to deliver therapeutic preparation 106 into a GI lumen wall or surrounding tissue of a subject.

Device 100 may be disposed in an enclosure 108 to allow for ingestion of device 100 in a GI tract of a subject. Enclosure 108 may include an outer coating 110, such as an enteric coating, to, for example, help prevent premature delivery of therapeutic preparation 106 and/or to temporarily protect the contents of device 100 while traversing a portion of the GI tract.

According to a non-limiting example, enclosure 108 (or optionally enclosure 108 and/or outer coating 110) can degrade at a desired location in the GI tract (e.g., the stomach or small intestine) of a subject for delivery of therapeutic preparation 106. In response to at least partial degradation of enclosure 108 and/or outer coating 110, first actuation mechanism 103 may generate the first force within expandable component 102 to cause expandable component 102 to expand. Expansion of expandable component 102 within the GI lumen causes delivery system 104 to be positioned relative to (e.g., adjacent or in contact with) a surface of the GI lumen wall. In response to expansion of expandable component 102, second actuation mechanism 105 may be actuated to generate the second, local force within delivery system 104 to deliver therapeutic preparation 106 into the GI lumen wall or surrounding tissue thereof.

Enclosure 108 and/or outer coating 110 are structured to allow for ingestion of device 100, and to temporarily protect the contents of device 100 from degradation within one or more portions of the GI tract of a subject. Enclosure 108 may take a variety of different forms and shapes, such as a swallowable capsule (e.g., a size 00 capsule, a size 000 capsule, or other size capsule), an endoscope, a stomach port, or any other structure that is suitable for ingestion and can house or contain one or more of the components of device 100. In one or more embodiments, enclosure 108 includes two or more sections coupled (e.g., press-fit) together to define enclosure 108. For example, enclosure 108 may be structured as a capsule including a first section at least partially overlapping a second section in a press-fit arrangement to define enclosure 108. The first and second sections may be detachably coupled together so as to allow for separation of the two sections.

In one or more embodiments, enclosure 108 (or optionally enclosure 108 and/or outer coating 110) can degrade under certain conditions. Further, different portions of enclosure 102 may be structured to degrade under different conditions or at different degradation rates depending on a target site within the GI tract for delivering therapeutic preparation 106. For example, a portion of, or all of, enclosure 108 may be constructed of a material that degrades in water (e.g., in the presence of water in the form of humidity or moisture in an ambient environment, such as within the body) and/or degrades when exposed to solutions with a pH level above a particular threshold or within a particular range (e.g., a pH level associated with a desired location or portion of the GI tract). In other embodiments, enclosure 108 degrades in response to a temperature threshold.

Outer coating 110 optionally covers a portion of, or all of, enclosure 108 or expandable component 102/delivery system 104. Outer coating 110 may include a single layer or multiple layers. The various layers may be formed of the same material or a combination of different materials. An example of outer coating 110 is an enteric coating, such as an enteric coating that degrades in water at a given rate and/or degrades when exposed to solutions with a pH level above a particular threshold or within a particular range. Another example of outer coating 110 is a protective coating (e.g., wax), such as a coating which protects a portion of an outer surface of enclosure 108 from coming into contact with fluids or tissues (e.g., bodily tissue or fluids).

In one or more embodiments, degradation of enclosure 108 and/or outer coating 110 (if present) allows fluid (e.g., bodily fluid in the stomach or in the intestine) to enter into an interior of enclosure 108/outer coating 110 to trigger first actuation mechanism 103 of expandable component 102. Enclosure 102 and/or outer coating 110 (if present) may define one or more degradation areas for localized degradation of enclosure 108 and/or outer coating 110 so as to, for example, allow for controlled degradation and separation of enclosure 108. For example, outer coating 110 may be selectively applied only to certain areas of enclosure 108 (e.g., on the ends of enclosure 108) to expose a selected portion of enclosure 108 (e.g., a middle portion of enclosure 108 between the ends), thereby defining an area of enclosure 108 that can degrade at a faster rate and/or degrade sooner than other areas of enclosure 108.

Expandable component 102 is a flexible and adjustable structure. In one or more embodiments, expandable component 102 is structured to expand from a collapsed state (e.g., folded, rolled, flattened) to an expanded state within a desired location of a GI lumen to locate and/or to align delivery system 104 relative to a surface of the GI lumen wall to facilitate delivery of therapeutic preparation 106 into the lumen wall. Expandable component 102 may have a variety of different shapes, sizes, and configurations for being temporarily stored in enclosure 108 and for being deployed within a lumen of the GI tract of a subject. Expandable component 102 may be structured as a balloon, a bellows/accordion structure, foldable wings, or any other structure that can adjust from a collapsed state to an expanded state to occupy a space within a GI lumen.

Expandable component 102 may define an interior volume for containing various components of device 100 including first actuation mechanism 103. For example, first actuation mechanism 103 may be structured as a gas generating mechanism including one or more substances that are temporarily separated from each other within an interior volume of expandable component 102 by a release mechanism. The interior volume may be structured to facilitate a chemical reaction caused by mixing the substances together (e.g., in response to activation of the release mechanism) to form a gas to cause expansion of expandable component 102. The gas pressure generated within expandable component 102 may be applied externally to delivery system 104 to initiate actuation of second actuation mechanism 105 in a sequential, multi-stage manner.

In another example, first actuation mechanism 103 includes a mechanical actuator to generate a mechanical force. For example, first actuation mechanism 103 may include a spring mechanism or similar biasing component structured to change from a pre-loaded (e.g., compressed) state to an expanded state. The spring mechanism may be temporarily held in a compressed state by a release mechanism, such as a biodegradable release. The spring mechanism may be used to expand expandable component 102 and to initiate actuation of second actuation mechanism 105. Alternatively, the spring mechanism may be structured to apply an external force to delivery system 104 to initiate actuation of second actuation mechanism 105 and a different mechanism (e.g., a gas generating mechanism) may be used to cause expansion of expandable component 102. The spring mechanism may be disposed within, or otherwise coupled to, expandable component 102.

In another example, first actuation mechanism 103 may include a hydrogel that can expand upon contact with a fluid, such as a bodily fluid in the GI tract of a subject. The hydrogel may be used to apply an external force to delivery system 104 to initiate actuation of second actuation mechanism 105.

Expandable component 102 may further include a feature for collapsing (e.g., deflating) expandable component 102 upon delivery of therapeutic preparation 106 to facilitate excretion of device 100 from a subject. Expandable component 102 is structured to stretch a defined amount without perforation upon expansion.

Expandable component 102 may be formed from one or more materials. Examples of suitable materials for expandable component 102 include, but are not limited to, hydroxypropyl methylcellulose (HPMC), polyvinyl acetate (PVA), lactide, glycolide, lactic acid, glycolic acid, par-dioxanone, trimethylene carbonate, caprolactone, and mixtures and copolymers thereof. Expandable component 102 may include one or more layers of material. Expandable component 102 may be a monolithic structure. In other embodiments, expandable component 102 may be composed of one or more sections that are coupled (e.g., sealed) together. Expandable component 102 may be formed at least partially, or fully, from a biodegradable material to allow for degradation of at least a portion of expandable component 102 within a GI tract of a subject upon delivery of therapeutic preparation 106.

Delivery system 104 is a chemical, mechanical, electrical, electro-mechanical, electro-chemical, chemo-mechanical, or electro-mechanical-chemical structure. In one or more embodiments, delivery system 104 includes, among other components, a housing 107, second actuation mechanism 105, and therapeutic preparation 106. Delivery system 104 is structured to be coupled to expandable component 102. Delivery system 104 may have a modular design to allow for aseptic assembly with therapeutic preparation 106 before coupling to expandable component 102. In response to an external force, second actuation mechanism 105 is structured to generate a second, local force within housing 107 to deliver therapeutic preparation 106 into the GI lumen wall or surrounding tissue. Device 100 may include one or more delivery systems 104 coupled to expandable component 102 to allow for the delivery of one or more therapeutic preparations. Alternatively, delivery system 104 itself may be structured to deliver a plurality of therapeutic preparations to thereby provide a desired therapeutic effect.

Housing 107 is a mechanical structure that includes one or more chambers for containing second actuation mechanism 105 and therapeutic preparation 106. For example, housing 107 may include a first chamber for containing second actuation mechanism 105 and a second chamber for containing therapeutic preparation 106. A delivery component, such as a piston, may be movably disposed within housing 107. Second actuation mechanism 105 may generate a local force within the first chamber to cause the delivery component to deliver therapeutic preparation 106 from the second chamber into the GI lumen wall.

Second actuation mechanism 105 is a chemical, mechanical, electrical, electro-mechanical, electro-chemical, chemo-mechanical, or electro-mechanical-chemical structure. Second actuation mechanism 105 is structured to generate a local force within housing 107 in response to a force applied to delivery system 104 to deliver therapeutic preparation 106 into the GI lumen wall or surrounding tissue. For example, second actuation mechanism 105 may be structured as a gas generating mechanism including one or more substances that are temporarily separated from each other within housing 107 (e.g., within the first chamber) by a separator (e.g., a pierceable film, a substrate with a detachable plug, a valve member). Housing 107 may be structured to facilitate a chemical reaction caused by mixing the substances together to form a gas within housing 107 to cause the delivery component to deliver therapeutic preparation 106 from housing 107 into the GI lumen wall or surrounding tissue. Other gas generating mechanisms may be used within housing 107, such as a pressurized gas cylinder.

According to another example, second actuation mechanism 105 may be structured as a mechanical actuator to generate a local mechanical force within housing 107 to cause the delivery component to deliver therapeutic preparation 106 into the GI lumen wall. For example, second actuation mechanism 105 may include a spring mechanism or similar biasing component disposed within housing 107. The spring mechanism may be structured to expand from a pre-loaded (e.g., compressed) state to cause the delivery component to eject therapeutic preparation 106 into a GI lumen wall.

Therapeutic preparation 106 includes one or more components and is intended for a therapeutic, diagnostic, or other biological purpose. Therapeutic preparation 106 may be in a solid or a fluid form, which fluid form may include a liquid, a slurry, a gel, a colloidal suspension, a gas, a powder, or any combination thereof. Therapeutic preparation 106 includes one or more components including one or more therapeutic agents, such as a drug (e.g., immunosuppressive drugs (e.g., adalimumab, ustekinumab), chemotherapy drugs, central nervous system (CNS) drugs (e.g., antiparkinson agents, antiemetic agents), antidiabetic drugs (e.g., metformin)), a protein, a peptide, a polypeptide, an antibody, a hormone (e.g., parathyroid hormone (PTH), follicle stimulating hormone (FSH)), an incretin or a combination thereof (e.g., GLP-1, GLP-2, GIP, glucagon, PYY, and analogues thereof)), an enzyme replacement therapy (ERT), an oligonucleotide (e.g., antisense oligonucleotides (ASO), RNA interference (RNAi), aptamer RNAs), a DNA or SiRNA transcript, a cell, a cytotoxic agent, a vaccine or other prophylactic agent, a nutraceutical agent, a vasodilator, or a vasoconstrictor, a delivery enhancing agent, a delay agent, an excipient, a diagnostic agent, or a substance for cosmetic enhancement. Therapeutic preparation 106 may include a therapeutically effective amount of one or more therapeutic agents as well as suitable amounts of other components (e.g., excipients) to achieve a desired therapeutic effect in a subject.

One or more components of device 100 (e.g., expandable component 102, delivery system 104, enclosure 108, outer coating 110) may be formed from, or otherwise include, one or more biodegradable materials to facilitate degradation of such components within a GI tract of a subject to, for example, allow for passage through the remainder of the intestinal tract of the subject after delivery of therapeutic preparation 106. Examples of biodegradable materials that may be suitable for use with various components of device 100 include, for example, hydroxypropyl methylcellulose (HPMC), polyvinyl acetate (PVA), lactide, glycolide, lactic acid, glycolic acid, par-dioxanone, trimethylene carbonate, caprolactone, and mixtures and copolymers thereof.

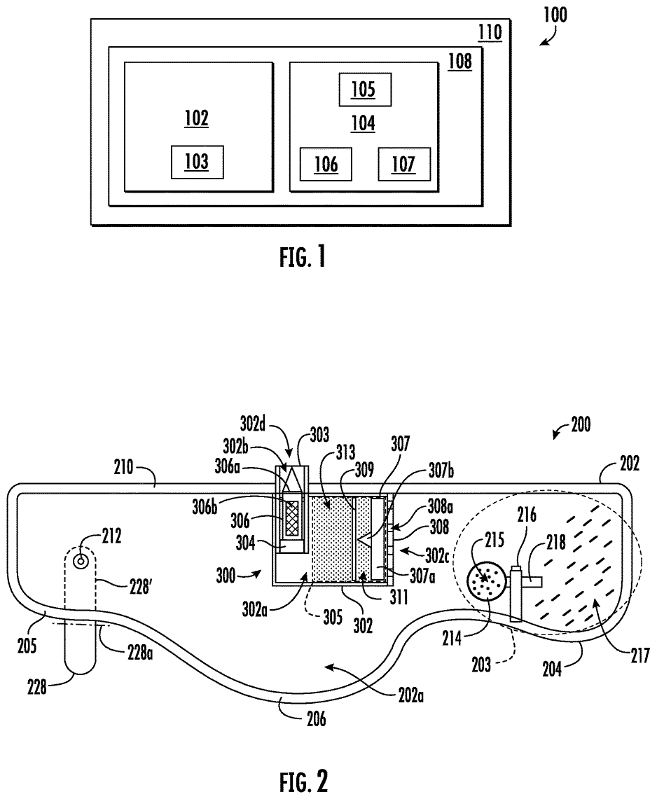

The preceding description is an overview of ingestible device 100. The following description with reference to FIGS. 2-6 relates to examples of various features and aspects of device 100. These examples are illustrative of, and not limiting on, device 100.

Referring to FIG. 2, a cross-sectional view of a device 200 (an embodiment of device 100) is illustrated in an unfolded and/or unrolled state. Device 200 is illustrated without an enclosure and/or outer coating. However, it should be appreciated that device 200 may be folded, rolled, and/or otherwise manipulated to be disposed within an enclosure and/or outer coating to allow for ingestion of device 200 and subsequent delivery into a GI tract of a subject.

Device 200 includes an expandable component in the form of a balloon 202 (an embodiment of expandable component 102), a delivery system 300 (an embodiment of delivery system 104) coupled to balloon 202, and a therapeutic preparation 306 (an embodiment of therapeutic preparation 106) disposed in delivery system 300.

Balloon 202 is shown to include an actuation mechanism in the form of a first gas generating mechanism 203 (an embodiment of first actuation mechanism 103). First gas generating mechanism 203 includes a reactant reservoir 214 containing a first substance 215, a release 216, and a second substance 217. Balloon 202 further includes a deflation mechanism 212.

Still referring to FIG. 2, balloon 202 includes an inflator section 204, a deflator section 205, a lower section 206, and an elongated section 210 extending between inflator section 204 and deflator section 205. Lower section 206 extends downward (in the orientation shown in FIG. 2) between inflator section 204 and deflator section 205. The various sections of balloon 202 cooperatively define an interior 202a for containing various components of device 200. As discussed below, balloon 202 is structured to inflate at a desired location within a lumen of a GI tract (e.g., stomach, small intestine, large intestine) in response to the formation of a gas from a chemical reaction between first substance 215 and second substance 217 within interior 202a. In this way, balloon 202 can locate and/or orient delivery system 300 relative to a surface of the GI lumen wall to facilitate delivery of therapeutic preparation 306.

Balloon 202 has a size and shape to occupy a space in a GI lumen upon inflation of balloon 202 to allow for delivery of therapeutic preparation 306 into the GI lumen wall. For example, upon inflation of balloon 202, an outer periphery of balloon 202 (e.g., an outer periphery of lower section 206 and elongated section 210) pushes against a surface of the lumen wall. The pressure exerted by balloon 202 is sufficient to temporarily hold balloon 202 relative to the lumen wall for delivery of therapeutic preparation 306. Depending on an inner circumference of the lumen delivery site, lower section 206 may remain partially folded, or may extend fully, when balloon 202 is inflated. For example, if the lumen is relatively large and there is no obstruction to resist expansion of balloon 202, then balloon 202 would assume a fully inflated configuration with lower section 206 fully extended (as shown in FIG. 2). If, however, the lumen is relatively small such that the inner lumen circumference is less than a maximum dimension of the fully inflated balloon 202, then lower section 206 would remain partially folded. In this way, balloon 202 can self-adjust to the size of a GI lumen to hold balloon 202 in position for delivery of therapeutic preparation 306, such that the same balloon 202 can be used for a broad range of lumen sizes (e.g., different inner circumferences).

Deflation valve 212 is structured to cause deflation of balloon 202 upon completion of delivery of therapeutic preparation 306 into the GI lumen wall or surrounding tissue thereof. In this way, deflation valve 212 can facilitate passage of balloon 202 through the remainder of the GI tract to exit the anus of the subject. In the embodiment shown, deflation valve 212 is structured as a degradable plug which temporarily covers an opening leading into interior 202a. The degradable plug may be structured to degrade in response to contact with fluid in the GI tract (e.g., bodily fluid) to thereby allow gas contained in interior 202a to exit through the opening. For example, deflation valve 212 may be formed from, or include, an enteric material. Balloon 202 may include an optional flap 228 which can temporarily cover deflation valve 212 until balloon 202 is inflated to thereby prevent premature activation (e.g., degradation) of deflation valve 212. For example, flap 228 may be temporarily held (e.g., adhered, tacked, or otherwise held) in a folded position 228′ about a flap folding axis 228a. The expansion of balloon 202 can cause flap 228 to unfold from the folded position 228′ to expose deflation valve 212 and allow fluid in the GI tract to reach deflation valve 212 for subsequent activation.

Deflation valve 212 is shown located on deflator section 205, but deflation valve 212 may be located elsewhere on expandable member 202 according to other embodiments. Further, balloon 202 may include more than one deflation valve 212. Deflation valve 212 may be structured differently than the embodiment shown in FIG. 2. For example, in other embodiments, deflation valve 212 may be structured as a degradable and/or movable cover disposed over an opening on balloon 202. In these embodiments, degradation and/or movement of the cover away from the opening can cause gas to exit from interior 202a through the opening.

Reactant reservoir 214 is disposed within interior 202a and is structured to hold first substance 215 therein and to temporarily prevent first substance 215 from contacting second substance 217, which is separately disposed within interior 202a. First substance 215 may be, for example, citric acid. Second substance 217 may be, for example, a carbonate, such as potassium bicarbonate. In other embodiments, first substance 215 and second substance 217 may be other types of reactants (e.g., an acid and a base) which when mixed result in the formation of a gas sufficient to inflate balloon 202. Second substance 217 is shown disposed within interior 202a at inflator section 204 near first substance 215. In other embodiments, first substance 215 and second substance 217 may be contained in other areas of balloon 202 so long as they are temporarily separated from each other.

Reactant reservoir 214 defines an interior volume for containing first substance 215. Reactant reservoir 214 may take a variety of different forms and shapes, such as a balloon or other structure. Reactant reservoir 214 is in selective fluid communication with interior 202a via reactant conduit 218 and release 216. Release 216 is coupled to reactant conduit 218 such that upon activation (e.g., degradation) of release 216, first reactant 215 can exit from reactant reservoir 214 into interior 202a via reactant conduit 218. For example, release 216 may be in the form of a biodegradable plug which blocks an interior portion of reactant conduit 218 to temporarily prevent first reactant 215 from entering interior 202a. When fluid in the GI tract contacts release 216 (e.g., upon degradation of enclosure 102 and/or outer coating 104), release 216 can subsequently degrade to allow reactant conduit 218 to discharge first reactant 215 into interior 202a. In these embodiments, release 216 may be formed from an enteric material or other biodegradable material that can degrade upon contact with a fluid (e.g., water, GI fluid).

In other embodiments, device 200 may include a clip, a band, or other structure for holding a portion of balloon 202 in such a manner to temporarily define separate chambers within interior 202a for separately containing first substance 215 and second substance 217, respectively. For example, a portion of balloon 202 may be pinched or compressed by a degradable clip or band to temporarily define the separate chambers within interior 202a. The chambers may be substantially sealed from each other to substantially prevent first substance 215 and second substance 217 from mixing. The clip or band may be located on an outer portion of balloon 202 such that exposure to fluid in the GI tract (e.g., upon degradation of enclosure and/or outer coating) can cause degradation of the clip or band and subsequent release from balloon 202. Upon release from balloon 202, the separate chambers can be combined to allow first substance 215 to mix with second substance 217 within interior 202a.

Combining first substance 215 with second substance 217 within interior 202a causes a chemical reaction resulting in the formation of a gas (e.g., CO₂.) The gas causes balloon 202 to expand to an inflated state within a GI lumen resulting in substantial alignment of elongated section 210 with a surface of the GI lumen wall. Substantial alignment of elongated section 210 relative to the GI lumen wall can, advantageously, help to facilitate delivery of therapeutic preparation 306 from delivery system 300 into the GI lumen wall or surrounding tissue thereof for systemic uptake of one or more therapeutic agents contained in therapeutic preparation 306.

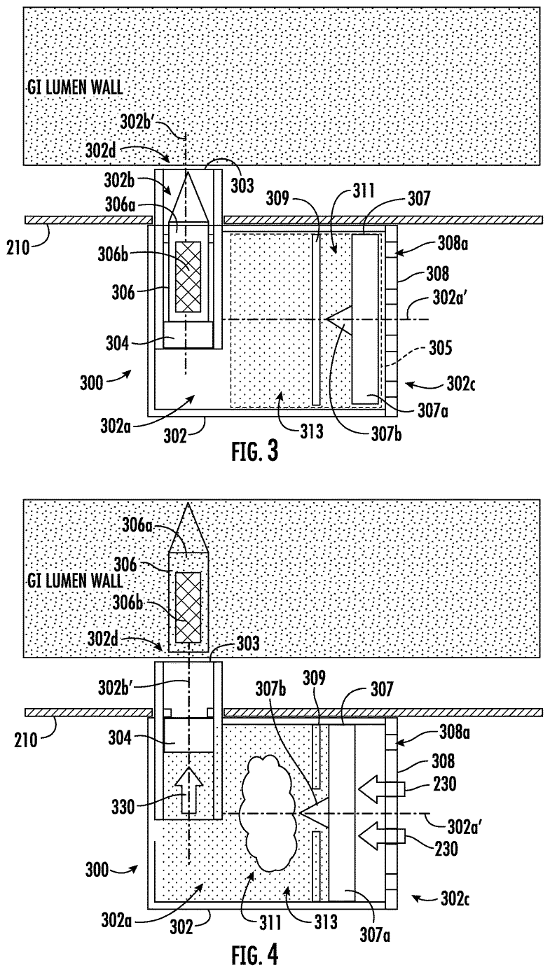

Referring to FIGS. 2-3, delivery system 300 includes a housing 302 (an embodiment of housing 107), a second actuation mechanism in the form of a second gas generating mechanism 305 (an embodiment of second actuation mechanism 105), a delivery component in the form of a delivery piston 304, and therapeutic preparation 306.

As shown in FIGS. 2-3, housing 302 is coupled to balloon 202 at elongated section 210 and is partially disposed within interior 202a. In other examples, housing 302 may be disposed entirely inside balloon 202 or entirely outside of balloon 202. Housing 302 includes a first chamber 302a and a second chamber 302b. Housing 302 has a substantially cylindrical shape where first chamber 302a is oriented perpendicularly relative to second chamber 302b, although other shapes (e.g., cuboidal, egg shape) and relative orientations (e.g., non-perpendicular) are contemplated according to other embodiments. First chamber 302a defines a first axis 302a′ and second chamber 302b′ defines a second axis 302b′. First axis 302a′ is oriented laterally and second axis 302b′ is oriented longitudinally in the orientation shown in FIGS. 2-3. First chamber 302a extends from second chamber 302b to a proximal end 302c. Proximal end 302c defines an opening for receiving a gas pressure from within interior 202a of balloon 202 to initiate actuation of second gas generating mechanism 305 in a sequential, multi-stage manner. Second chamber 302b extends outwardly to a distal end 302d from a periphery of elongated section 210. Distal end 302d defines an opening for delivery of therapeutic preparation 306 from housing 302 into the GI lumen wall.

A cover 308 is shown coupled (e.g., adhered, heat sealed) to housing 302 at proximal end 302c. Cover 308 includes openings 308a to provide a fluid path between interior 202a of balloon 202 and second gas generating mechanism 305 to allow for actuation of second gas generating mechanism 305. As discussed below, cover 308 may also function as a stop for limiting the axial movement of an actuation piston 307 which forms part of second gas generating mechanism 305.

A seal 303 is coupled at distal end 302d to temporarily cover the opening to second chamber 302b and to protect therapeutic preparation 306 from exposure to the GI environment. For example, seal 303 may be a foil (e.g., aluminum foil) adhered to housing 302 at distal end 302d. Seal 303 may be structured to tear, be punctured, or otherwise open upon contact with therapeutic preparation 306 to permit therapeutic preparation 306 to exit second chamber 302b to penetrate a GI lumen wall, the details of which are discussed in the paragraphs that follow.

Second gas generating mechanism 305 is disposed in first chamber 302a and includes an actuation piston 307, a separator in the form of a pierceable membrane 309, a first substance 311, and a second substance 313. First substance 311 and second substance 313 may be the same as, or different from, first substance 215 and second substance 217 in balloon 202, respectively.

Actuation piston 307 includes a piston 307a and a piercing member 307b extending outwardly from a surface of piston 307a. Piercing member 307b may be coupled to, or integrally formed with, piston 307a. Piercing member 307b has a conical shape and terminates at a distal tip that is structured to puncture pierceable membrane 309. Piercing member 307b may have other shapes and configurations, such as a needle shape or other shape sufficient to pierce, puncture, tear, or otherwise create an opening through pierceable membrane 309. Actuation piston 307 is movably disposed within first chamber 302a with piercing member 307b facing inwardly toward second chamber 302b and is structured to move axially along first axis 302a. For example, actuation piston 307 may be structured to slide within first chamber 302a along an interior surface of housing 302. Actuation piston 307 may include a seal (e.g., a silicone seal) disposed between housing 302 and actuation piston 307 to create a substantially fluid tight seal within first chamber 302a to thereby contain a substantial amount of the gas generated by second gas generating mechanism 305 within housing 302. Cover 308 can also function to limit the axial movement of actuation piston 307 relative to housing 302 upon second gas generating mechanism 305 generating a second gas pressure within first chamber 302a to thereby help to substantially contain the gas pressure within housing 302.

In other embodiments, housing 302 may include a separate stop feature (e.g., a flange, a separate seal) located at or near proximal end 302c of first chamber 302a away from second chamber 302b. The stop feature can function to limit axial movement of actuation piston 307 and/or help to create a substantially fluid tight seal within first chamber 302a to thereby substantially contain the gas pressure generated therein.

Actuation piston 307 may be structured to move relative to housing 302 in response to the gas pressure generated within interior 202a of balloon 202 reaching or exceeding a first threshold pressure (e.g., a first threshold value, an actuation threshold). The first threshold pressure may be associated with an inflated (partially or fully) state of balloon 202 such that delivery system 300 is positioned in proximity to a surface of a GI lumen wall for delivery of therapeutic preparation 306 before movement of actuation piston 307 is initiated. For example, the first threshold gas pressure generated within interior 202a may be in a range of about 5 psi to about 20 psi.

In one example, actuation piston 307 may be temporarily held in position relative to housing 302 by a foil (e.g., aluminum foil) coupled to housing 302 and piston 307. The foil may include a local stress concentration, a local thickness reduction, or otherwise be structured to tear, separate, or detach from piston 307 in response to the gas pressure generated in interior 202a reaching or exceeding the first threshold pressure to thereby allow axial movement of piston 307 relative to housing 302.

In another example, actuation piston 307 may be temporarily held in position relative to housing 302 by a deformable component coupled to housing 302. The deformable component may be structured to create an interference condition with actuation piston 307. The deformable component may include features (e.g., protrusions) that are structured to deflect, move, or otherwise permit actuation piston 307 to overcome the interference condition with deformable component upon the gas pressure in interior 202a reaching or exceeding the first threshold pressure to thereby allow axial movement of actuation piston 307 relative to housing 302.

In another example, actuation piston 307 may be detachably coupled to housing 302 by a frangible connection that is designed to fail (e.g., break, separate) at or above the first threshold gas pressure to allow axial movement of actuation piston 307. For example, actuation piston 307 may be detachably coupled to housing 302 by one or more breakable fingers extending from housing 302. The one or more breakable fingers may be frangibly connected to a surface of actuation piston 307. Upon the gas pressure in interior 202a reaching or exceeding the first threshold pressure, the one or more breakable fingers may fail or otherwise detach from actuation piston 307 to allow axial movement of actuation piston 307 relative to housing 302.

Pierceable membrane 309 is coupled to housing 302 within first chamber 302a. For example, pierceable membrane 309 may include a foil (e.g., aluminum foil) that is adhered to an inner surface of housing 302 within first chamber 302a. Pierceable membrane 309 is structured to temporarily fluidly separate first substance 311 from second substance 313 within first chamber 302a. Pierceable membrane 309 is further structured to be pierced, punctured, torn, detached, or otherwise opened to provide a fluid path therethrough by piercing member 307b to thereby allow first substance 311 to mix with second substance 313 in response to axial movement of actuation piston 307.

In other embodiments, instead of using pierceable membrane 309 and piston 307a with piercing member 307b, delivery system 300 may include a separator in the form of a membrane that is structured to detach, tear, or otherwise open to provide a fluid path therethrough in response to a pressure (e.g., fluidic pressure) applied by piston 307a and first substance 311 against the membrane without a piercing member 307b. For example, the membrane may include a foil having a local stress point (e.g., a localized thickness variation, an etched area on the foil, an additional material applied to a portion of the foil) designed to cause tearing or detaching of the foil in response to a fluidic pressure applied by piston 307a and first substance 311. The piston 307a may move axially along first axis 302a′ toward the membrane to compress first substance 311 against the foil. The pressure applied by first substance 311 may cause the foil to tear or detach from housing 302 and thereby provide a fluid path through the membrane for combining first substance 311 with second substance 313.

As shown in FIGS. 2-3, first substance 311 is disposed between pierceable membrane 309 and actuation piston 307. First substance 311 may be in a fluid form (e.g., a liquid, a gel). For example, first substance 311 may be an acid, such as citric acid. First substance 311 may be disposed directly in first chamber 302a. Alternatively, first substance 311 may be disposed in a pouch or other container within first chamber 302a. The pouch or container may be structured to be punctured or otherwise opened by piercing member 307b to expel first substance 311.

Second substance 313 is disposed in first chamber 302a on an opposite side of pierceable membrane 309 separate from first substance 311, although it should be appreciated that the locations of first substance 311 and second substance 313 within first chamber 302a may be reversed according to other examples. Second substance 313 may be in a powder or a solid form, such as a tablet. For example, second substance 313 may be a carbonate, such as potassium bicarbonate. Second substance 313 is configured to react with first substance 311 to generate a gas (e.g., CO₂) within housing 302. Second substance 313 may be disposed directly in first chamber 302a. Alternatively, second substance 313 may be disposed in a pouch or other container within first chamber 302a.

Delivery piston 304 is slidably disposed in housing 302 between first chamber 302a and second chamber 302b. Delivery piston 304 is structured to move axially along second axis 302b′ toward therapeutic preparation 306 in response to a local gas pressure (e.g., a second gas pressure) generated within first chamber 302a. Delivery piston 304 is structured to engage a surface of therapeutic preparation 306 to eject therapeutic preparation 306 from second chamber 302b through seal 303 into a GI lumen wall upon the local gas pressure within first chamber 302a reaching or exceeding a second threshold gas pressure (e.g., a second threshold value, a delivery pressure). For example, the second threshold pressure may be in a range of about 25 psi to about 250 psi.

In one example, delivery piston 304 may have an interference fit with housing 302, such as via a protrusion or other feature, or with an intermediate component disposed in housing 302, such as a seal. In this way, movement of delivery piston 304 relative to housing 304 occurs when the second gas pressure created within first chamber 302a is sufficient to cause delivery piston 304 to overcome the interference condition with housing 302. Delivery system 300 may be structured such that the second threshold gas pressure is sufficient to cause delivery piston 304 to force therapeutic preparation 306 from second chamber 302b to a desired penetration depth in the GI lumen wall or in the surrounding tissue (e.g., the peritoneum or peritoneal cavity).

Therapeutic preparation 306 is disposed in second chamber 302b. Therapeutic preparation 306 includes a needle 306a and a tablet 306b disposed in a cavity of needle 306a. Needle 306a has a generally elongated shape with a tapered or pointed tip structured to penetrate a GI lumen wall. In other examples, needle 306a may have a hook or other structure sufficient to pierce GI lumen wall tissue. Needle 306a is formed from or comprises a substantially biodegradable material that is structured to degrade at least in part within a GI lumen wall or surrounding tissue of a GI tract of a subject to expose tablet 306b to a subject's bodily environment (e.g., bodily tissue, bodily fluid) for release of the therapeutic agent. For example, needle 306a may be formed from, or otherwise include, one or more biodegradable materials, such as a sugar (e.g., maltose), polyethylene oxide (PEO), magnesium, or other biodegradable material or combinations of materials. Needle 306a may include a cavity therein for holding tablet 306b, which may contain one or more therapeutic agents. For example, tablet 306b may be a compressed tablet formed from a lyophilized powder formulation of one or more therapeutic agents and one or more excipients or other components. Tablet 306b may be structured to degrade in the GI lumen wall or surrounding tissue to release the therapeutic agent into the subject's blood stream to provide a desired therapeutic effect. Tablet 306b may be formulated to include a dosage of up to about 5 mg of one or more therapeutic agents.

In other embodiments, instead of using a tablet 306b in the cavity of needle 306a, the therapeutic agent may be in the form of a fluid disposed in the cavity of needle 306a. For example, the therapeutic agent may be a liquid or a gel formulation disposed in the cavity.

In other embodiments, tablet 306b is formed into the shape of a needle (e.g., by compression in a mold that is shaped as a needle) without a separate biodegradable needle 306a to define therapeutic preparation 306. In these embodiments, tablet 306b may be structured to directly engage delivery piston 304 such that delivery piston 304 applies a force to tablet 306b to eject tablet 306b from second chamber 302b and penetrate a GI lumen wall. For example, tablet 306b may include a surface that is complementary to an engagement surface of delivery piston 304, such that delivery piston 304 can apply a force (e.g., a mechanical force) directly to tablet 306b to thereby eject tablet 306b from second chamber 302b through seal 303 into a GI lumen wall.

Referring to FIG. 3, delivery system 300 is shown in a first stage when balloon 202 is inflated at a desired location within a GI lumen (e.g., a stomach, an intestine) such that delivery system 300 is positioned proximate to the GI lumen wall. As shown in FIG. 3, expansion of balloon 202 within the GI lumen results in distal end 302d of housing 302 being positioned adjacent to a surface of the GI lumen wall and device 200 oriented such that second axis 302b′ is oriented substantially orthogonal to the surface of the GI lumen wall. At the stage shown in FIG. 3, the gas pressure generated within interior 202a has not yet reached the first threshold pressure sufficient to trigger second actuation mechanism 305.

Referring to FIG. 4, delivery system 300 is shown in a second stage when the first gas pressure generated within interior 202a of balloon 202 has reached or exceeded the first threshold gas pressure (represented by directional arrows 230). As shown in FIG. 4, the first gas pressure from within interior 202a is applied against a surface of actuation piston 307 through openings 308a to cause actuation piston 307 to overcome the interference condition with housing 302 and move axially along first axis 302a′ toward pierceable membrane 309. Upon sufficient axial movement of actuation piston 307, piercing member 307b pierces through pierceable membrane 309 to provide a fluid path for first substance 311 to mix with second substance 313 resulting in the formation of a second gas pressure within first chamber 302a which is applied against a surface of delivery piston 304.

Upon the second gas pressure 330 reaching or exceeding a second threshold gas pressure (represented by directional arrow 330) (e.g., a pressure associated with a desired penetration depth of therapeutic preparation 306), the second gas pressure causes delivery piston 304 to move axially along second axis 302b′ to engage therapeutic preparation 306 and eject therapeutic preparation 306 from second chamber 302b through seal 303 into the GI lumen wall or surrounding tissue, where needle 306a can degrade to release the one or more therapeutic agents contained in tablet 306b for systemic uptake.

Upon completing delivery of therapeutic preparation 306, one or more components of device 200 (e.g., balloon 202) can subsequently degrade within the GI lumen wall or surrounding tissue, or other area within the GI tract. Further, deflation valve 212 can release a substantial amount of the gas contained within interior 202a to allow for substantial deflation of balloon 202 and subsequent traversal of the device through the remainder of the GI tract to exit the anus of the subject.

Additionally, device 200 may include one or more vent pathways that fluidly connect first chamber 302a of delivery system 300 to the GI lumen environment to function as a gas pressure relief. For example, the vent pathway(s) may include a foil that is pierced or a plug that opens in response to the gas pressure generated in chamber 302a. The vent pathway(s) may be located along elongated section 210 near distal end 302c of delivery system 300.

The multi-stage actuator of device 200 can, advantageously, allow for the use of relatively small amounts of reactants (e.g., first substance 215 and second substance 217) in first gas generating mechanism 203 to inflate balloon 202. This is because the first gas pressure required to inflate balloon 202 can be significantly less than the second gas pressure required to deliver therapeutic preparation 306 into the GI lumen wall or surrounding tissue.

Moreover, due to the local pressure generation within delivery system 300, relatively small amounts of reactants (e.g., first substance 311 and second substance 313) can be used in second gas generating mechanism 305 to generate sufficiently high pressures for delivery of therapeutic preparation 306. Reducing the amounts of reactants in both first and second gas generating mechanisms 303,305 can, advantageously, allow for a reduction in device package size, which may help to facilitate use of a smaller enclosure 108 (e.g., a size 00 capsule) to orally deliver device 200.

In addition, the relatively lower balloon inflation pressure can allow for flexibility to, for example, use different materials for balloon 202, such as a fully biodegradable material, and/or a different material thickness for balloon 202 (e.g., a thinner balloon material due to the lower inflation pressure).

In addition, the relatively lower balloon inflation pressure can allow for improvements relating to device shelf life. For example, the reactants in device 200 may lose their gas generating capabilities over time during storage of the device. Thus, having a lower balloon inflation pressure means that less reactants are required to achieve sufficient inflation of balloon 202 to facilitate delivery of therapeutic preparation 306.

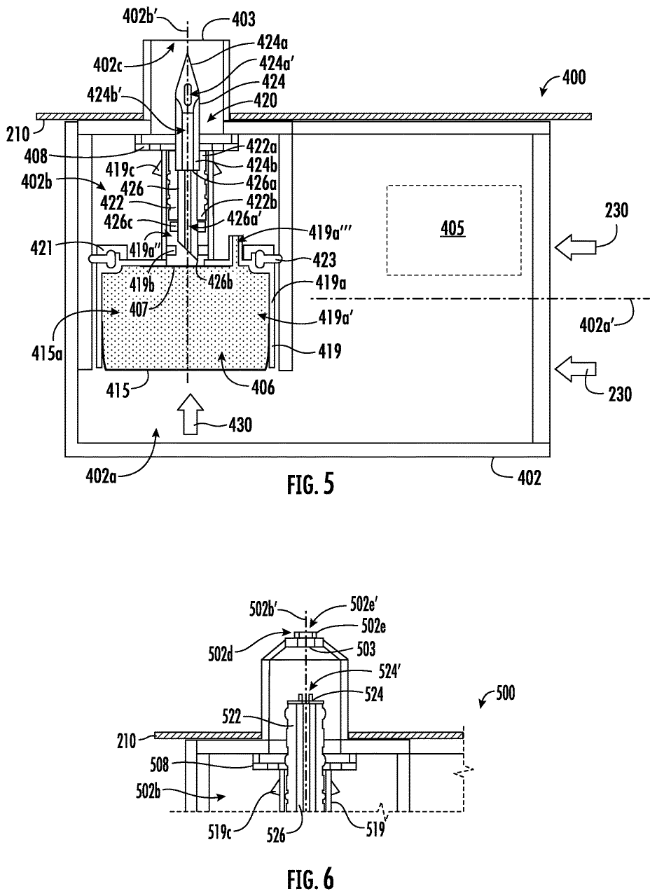

Referring now to FIG. 5, a delivery system 400 (another embodiment of delivery system 104) is shown coupled to elongated section 210 of balloon 202 according to another example. In this embodiment, delivery system 400 is structured to deliver a therapeutic preparation in the form of a fluid preparation 406 (another embodiment of therapeutic preparation 106). Delivery system 400 is shown to include a housing 402 (another embodiment of housing 107), a second actuation mechanism 405 (another embodiment of second actuation mechanism 105), a membrane 415, fluid preparation 406, a delivery piston 419, and a needle 420. Membrane 415 and delivery piston 419 cooperatively define a reservoir 415a for containing fluid preparation 406.

As discussed below, when the gas that pressurizes balloon 202 reaches or exceeds the first threshold gas pressure 230, second actuation mechanism 405 is actuated to generate a second gas pressure within first chamber 402a. When the second gas pressure reaches or exceeds a second threshold gas pressure (represented by directional arrow 430), the second gas pressure causes piston 419 and needle 420 to move axially relative to housing 402 toward the GI lumen wall. In response to a resistance applied to needle 420 from the GI lumen wall, piston 418 moves axially relative to needle 420 to cause needle 420 to pierce a seal on piston 419 to thereby allow fluid preparation 406 to flow from reservoir 415a to needle 420. Upon piercing the seal, the second gas pressure causes needle 420 to advance further into the GI lumen wall or surrounding tissue. The second gas pressure also causes membrane 415 to deform (e.g., constrict, compress) to expel fluid preparation 406 from reservoir 415a through needle 420 and into the GI lumen wall or surrounding tissue thereof.

Still referring to FIG. 5, delivery system 400 further includes a needle seal 403, a release mechanism 408, a reservoir seal 407, a piston seal holder 421, and a piston seal 423. In the embodiment shown, needle 420 is collectively defined by a needle sleeve 422, a tissue piercing member 424, and a seal piercing member 426. In other embodiments, needle 420 may be a monolithic structure.

Housing 402 is identical in structure to housing 302 discussed above and includes a first chamber 402a and a second chamber 402b. Like housing 302, first chamber 402a defines a first axis 402a′ and second chamber 402b defines a second axis 402b′. Second actuation mechanism 405 is shown schematically in first chamber 402a. However, it should be appreciated that second actuation mechanism 405 may be structured according to any of the example actuation mechanisms discussed herein.

Needle seal 404 is coupled (e.g., adhered) to housing 402 at a distal end 402c of housing 402 to substantially prevent fluid in the GI tract from entering into second chamber 402b until needle 420 (e.g., tissue piercing member 424) pierces needle seal 403. Needle seal 403 may be formed from a penetrable material (e.g., aluminum foil) to allow needle 420 to pierce through needle seal 403. In this way, needle seal 403 and housing 402 can cooperatively define a substantially sterile environment for containing needle 420 before delivery of fluid preparation 406 into the GI lumen wall or surrounding tissue.

Release mechanism 408 is coupled to (e.g., press-fit, snap-fit, adhered), or integrally formed with, housing 402 within second chamber 402b. As shown in FIG. 5, release mechanism 408 is coupled to an inner surface of housing 402 within second chamber 402b. Release mechanism 408 is structured to interface with piston 419 to substantially prevent advancement of piston 419 and needle 420 until sufficient pressure is generated by the second gas within first chamber 402a. For example, release mechanism 408 may include one or more fingers extending radially inward toward second axis 402b′. Each of the fingers may extend radially inward in a cantilevered manner from a circumferential edge of release mechanism 408 to a free end located adjacent piston 419. The fingers may be structured to interface with a protrusion 419c of piston 419 via an interference (e.g., overlapping) condition to substantially prevent needle 420 from piercing needle seal 403 until sufficient pressure is generated within first chamber 402a. When the second gas pressure reaches or exceeds the second threshold pressure 430, protrusion 419c overcomes the interference condition with the fingers of release mechanism 408 by causing the fingers to deflect away from piston 419 to thereby allow piston 419 to move axially along second axis 402b′ toward needle seal 403, such that tissue piercing member 424 pierces needle seal 403.

According to other examples, the release mechanism may be defined by a detachable connection between piston 419 and housing 402. For example, modular delivery system 400 may include a rear cover coupled to housing 402 between first chamber 402a and second chamber 402b. The rear cover may include one or more snap features which interface with a complementary feature on piston 419 to temporarily hold piston 419 relative to housing 402 until sufficient pressure is generated by the second gas within first chamber 402a.

In another example, the rear cover may have a frangible (e.g., breakable) connection with piston 419, which may be structured to detach in response to the second gas pressure generated within first chamber 402a to permit axial movement of piston 419 relative to housing 402.

Piston 419 is movably (e.g., slidably) coupled to housing 402 in second chamber 402b. Piston 419 includes a piston perimeter wall 419a which defines a fluid cavity 419a′ and a needle cavity 419a″. As discussed below, membrane 415 is coupled to piston 419 such that membrane 415 encloses fluid cavity 419a′ to define reservoir 415a for containing fluid preparation 406 therein. Piston 419 is structured to move axially along second axis 402b′ relative to housing 402 in response to the second gas pressure applied against an outer surface of membrane 315 reaching or exceeding the second threshold pressure 430. Piston perimeter wall 419a further defines a fill port 419a′″ for filling reservoir 415a with fluid preparation 406. After filling (partially or fully) reservoir 415a, fill port 419a′″ is substantially fluidly sealed by, for example, heat staking piston perimeter wall 419a to substantially block fill port 419a″′.

In another example, a separate seal (e.g., silicone or aluminum foil) may be coupled to piston perimeter wall 419a at fill port 419a″′.

In another example, fill port 419a″′ may include a septum (e.g., silicone septum) such that fill port 419a″′ can self-seal after filling reservoir 415a with fluid preparation 406. In either case, reservoir 415a may be filled with fluid preparation 406 before piston 419 is disposed in housing 402 and balloon 202. This can, advantageously, allow for flexibility relating to aseptic assembly of the device. For example, prior to assembling piston 419 with housing 402 and balloon 202, a vacuum may be applied to reservoir 415a via fill port 419a″′ in an aseptic environment to substantially evacuate reservoir 415a. The evacuated reservoir 415a can then be filled with fluid preparation 406 via fill port 419a″′ in the aseptic environment prior to assembly with housing 402 and balloon 202.

Still referring to FIG. 5, piston perimeter wall 419a further defines a stop feature 419b extending from an inner surface of piston perimeter wall 419a toward second axis 402b′ within needle cavity 419a″. Stop feature 419b is structured to interface with a complementary feature on needle 420 to limit the axial travel of piston 419 relative to needle 420, the details of which are discussed below.

Piston perimeter wall 419a further defines protrusion 419c extending from an outer surface thereof at an upper portion of needle cavity 419a″. In the unactuated state shown in FIG. 5, protrusion 419c is located adjacent release mechanism 408. As previously discussed, protrusion 419c interfaces with fingers of release mechanism 408 to substantially prevent advancement of needle 420 through needle seal 403 until the second gas pressure reaches or exceeds the second threshold pressure 430. For example, the second threshold pressure 430 may be selected based on a desired penetration depth of needle 420 for delivery of fluid preparation 406. For example, the second threshold value may be selected such that needle 420 penetrates into a peritoneum or peritoneal cavity of a subject for delivery of fluid preparation 406 into the peritoneum or peritoneal cavity.

In another example, the second threshold value may be selected such that needle 420 is inserted into a GI lumen wall (e.g., stomach wall or intestinal wall) of a subject for delivery of fluid preparation 406 into the lumen wall tissue (e.g., the mucosa, submucosa, muscularis externa, serosa).

Reservoir seal 407 is coupled (e.g., adhered) to an inner surface of piston perimeter wall 419a to define a substantially fluid-tight seal between fluid cavity 419a′ and needle cavity 419a″. In this way, reservoir seal 407 substantially prevents fluid preparation 406 from entering needle cavity 419a″ until needle 420 pierces reservoir seal 407. Reservoir seal 407 may be formed from a penetrable material such as aluminum foil.

Piston seal 423 is coupled to an outer portion of piston perimeter wall 419a by piston seal holder 421. Piston seal holder 421 is coupled (e.g., adhered, snap-fit) to an upper portion of piston perimeter wall 419a to hold piston seal 423 relative to piston 419. In other embodiments, piston seal 423 may be integrally formed with (e.g., insert molded), or otherwise coupled to, a peripheral side of piston 419. Piston seal 423 is structured to engage an inner surface of housing 402 to create a substantially fluid-tight seal between piston 419 and housing 402 within second chamber 402b. Piston seal 423 is further structured to allow for relative axial movement between piston 419 and housing 402 along second axis 402b′. Piston 419 may be formed from a polymeric material (e.g., ABS), or other material or combinations of materials. Piston seal 423 may be formed from silicone or other suitable material.

Still referring to FIG. 5, membrane 415 is coupled to piston 419 to define reservoir 415a. For example, membrane 415 may be coupled to piston 419 by heat sealing, ultrasonic welding, adhering, or by other means. In the embodiment shown, membrane 415 is coupled to an inner surface of piston perimeter wall 419a which defines fluid cavity 419a′. Membrane 415 extends circumferentially about piston 419 to enclose fluid cavity 419a′. In this way, membrane 415, piston perimeter wall 419a, and reservoir seal 407 cooperatively define reservoir 415a for containing fluid preparation 406 therein. Reservoir 415a may define a volume for containing up to about 250 μl or more of fluid. As discussed below, membrane 415 is structured to deform (e.g., bend, flex) in response to the second gas pressure generated within first chamber 402a to expel fluid preparation 406 from reservoir 415a to needle 420.

Membrane 415 may have a surface profile in a relaxed state (i.e., when reservoir 415a is unfilled with fluid preparation 406) that is complementary to an inner surface profile of piston 419 which defines fluid cavity 419a′. For example, membrane 415 may be vacuum formed using fluid cavity 419a′ as a mold for forming membrane 415 such that membrane 415 substantially mimics the inner surface profile of fluid cavity 419a′. In this way, membrane 415 can be sufficiently deformed to expel a substantial portion of fluid preparation 406 from reservoir 415a. Further, such a complementary shape of membrane 415 may help to facilitate sufficient evacuation of reservoir 415a for subsequent filling with fluid preparation 406. Membrane 415 may be formed from a flexible polymeric material (e.g., PET), or other flexible material or combinations of materials having sufficiently low moisture and gas permeability properties for use with fluid preparation 406.

Needle 420 is movably (e.g., slidably) coupled to piston 419 and is disposed in needle cavity 419a″. In the embodiment shown in FIG. 5, needle 420 is collectively defined by needle sleeve 422, tissue piercing member 424, and seal piercing member 426. In other embodiments, needle 420 is a monolithic structure.

Needle sleeve 422 couples tissue piercing member 424 to seal piercing member 426. Further, needle sleeve 422 functions to movably couple needle 420 to piston 419 within needle cavity 419a″. In the embodiment shown, needle sleeve 422 has a hollow cylindrical shape defining a needle sleeve first end 422a and an opposite needle sleeve second 422b. An outer surface of needle sleeve 422 engages with an inner surface of piston perimeter wall 419a within needle cavity 419a″ to define a substantially fluid-tight seal therebetween. In this manner, needle sleeve 422 can help to prevent fluid preparation 406 from passing through needle cavity 419a″ between needle 420 and piston perimeter wall 419a, such that a substantial amount of fluid preparation 406 can be directed from reservoir 415a through needle 420. Needle sleeve 422 is further structured to allow for relative axial movement between piston 419 and needle 420 along second axis 402b′. Needle sleeve 422 may be formed from, for example, silicone or other suitable material.

Tissue piercing member 424 is coupled (e.g., press-fit, adhered) to needle sleeve 422 at needle sleeve first end 422a. Tissue piercing member 424 extends outwardly from needle sleeve 422. Tissue piercing member 424 includes a tapered section 424a (e.g., pointed tip) which is structured to pierce though needle seal 403 and penetrate the GI lumen wall in response to sufficient axial movement of piston 419. The free end (e.g., pointed tip) of tapered section 424a defines a first end of needle 420. Tapered section 424a defines a needle opening 424a′ extending through a side of tapered section 424a for discharging fluid preparation 406 into the GI lumen wall or surrounding tissue. Tissue piercing member 424 further includes an elongated section 424b extending from tapered section 424a. Elongated section 424b may be coupled to, or integrally formed with, tapered section 424a. A portion of elongated section 424b is disposed in needle sleeve 422 at needle sleeve first end 422a. Elongated section 424b defines a first needle channel 424b′ extending from needle opening 424a′ to an opposite end of elongated section 424b. In other embodiments, elongated section 424b includes a side opening (e.g., radial opening) for discharging fluid preparation 406 into the GI lumen wall or surrounding tissue. Tissue piercing member 424 is at least partially, or fully, degradable such that at least a portion of tissue piercing member 424 can substantially degrade within the GI lumen wall (or other location in the GI tract) upon delivery of fluid preparation 406. For example, tissue piercing member 424 may be formed from, or include, PEO, magnesium, or other degradable material or combinations of materials.

Seal piercing member 426 is coupled to (e.g., press-fit, adhered) needle sleeve 422. In the embodiment shown, seal piercing member 426 is structured as a substantially cylindrical member with a generally elongated configuration, although other shapes and configurations are contemplated according to other embodiments. Seal piercing member 426 extends from a seal piercing member first end 426a to an opposite seal piercing member second end 426b. Seal piercing member second end 426b defines a second end of needle 420 located opposite the first end. Seal piercing member first end 426a is coupled to (e.g., press-fit, adhered) needle sleeve 422 at needle sleeve second end 422b. Seal piercing member 426 extends outwardly from needle sleeve second end 422b and terminates at seal piercing member second end 426b. Seal piercing member second end 426b has a tapered profile (e.g., pointed end) which is structured to pierce reservoir seal 407 in response to relative axial movement between piston 419 and needle 420. Seal piercing member 426 further defines a protrusion 426c extending outwardly (e.g., radially) from an outer surface thereof. As discussed below, protrusion 426c functions as a complementary feature for interfacing with stop feature 419b to limit the axial travel of piston 419 relative to needle 420. Seal piercing member 426 further defines a second needle channel 426a′ for directing fluid preparation 406 from reservoir 415a to first needle channel 424b′ of tissue piercing member 424. Second needle channel 426a′ extends longitudinally along second axis 402b′ from seal piercing member first end 426a to seal piercing member second end 426b. Seal piercing member 426 may be formed from, for example, a polymeric material.

The function of modular delivery system 400 will now be discussed when balloon 202 is inflated at a desired location of a GI tract of a subject for delivery of fluid preparation 406. The generated gas within interior 202a of balloon 202 applies a first gas pressure against second gas generating mechanism 405 at an opening of housing 402. Upon the first gas pressure reaching or exceeding the first threshold pressure 230 (e.g., a pressure associated with a partially or fully inflated state of balloon 202), second gas generating mechanism 405 is actuated to generate a second gas pressure within first chamber 402a.

When the second gas pressure reaches or exceeds the second threshold pressure 430, protrusion 419c overcomes the interference condition with release mechanism 408. As a result, piston 419 moves axially relative to housing 402 along second axis 402b′ toward the GI lumen wall such that the first end of needle 420 (e.g., tapered section 424a) is advanced through needle seal 403 to contact the GI lumen wall.

Upon sufficient resistance from the GI lumen wall acting on needle 420, the second gas pressure applied against membrane 415 causes piston 419 to move axially relative to needle 420 toward the GI lumen wall along second axis 402b′. For example, modular delivery system 400 may be structured such that piston 419 moves relative to needle 420 upon needle 420 initiating contact with the GI lumen wall to generate sufficient resistance with the lumen wall. In other embodiments, modular delivery system 400 may be structured such that piston 419 moves relative to needle 420 upon needle 420 penetrating into the GI lumen wall to generate sufficient resistance with the lumen wall. This may be achieved by, for example, selecting a material of piston 419 and/or of needle sleeve 422 to have a sufficient coefficient of friction to control relative movement between piston 419 and needle 420. In either case, fluid preparation 406 remains in reservoir 415a until the second end of needle 420 (e.g., seal piercing member second end 426b) pierces reservoir seal 407 while the gas pressure in balloon 202 is applied against membrane 415. In this manner, modular delivery system 400 can allow for sequential timing between penetrating the GI lumen wall and discharging fluid preparation 406, which may help to substantially avoid discharging fluid preparation 406 into the lumen environment and ensure delivery of fluid preparation 406 into the GI lumen wall or surrounding tissue.

As a result of sufficient movement of piston 419 relative to needle 420, the second end of needle 420 pierces reservoir seal 407 to allow fluid preparation 406 to flow from reservoir 415a to second needle channel 426a′. Piston 419 is permitted to move axially relative to needle 420 until stop feature 419b engages protrusion 426c. Upon engagement between stop feature 419b and protrusion 426c, the gas pressure applied against membrane 415 causes piston 419 and needle 420 to move axially together relative to housing 402 along second axis 402b′ such that needle 420 penetrates further into the GI lumen wall or surrounding tissue.

In addition, upon piercing reservoir seal 407, the gas pressure applied against membrane 415 causes membrane 415 to deform inward (e.g., constrict, compress) relative to piston 419 toward needle 420 to expel fluid preparation 406 from reservoir 415a to needle 420. As a result, fluid preparation 406 is directed through second needle channel 426a′, first needle channel 424b′, and needle opening 424a′ into the GI lumen wall or surrounding tissue. In this manner, one or more therapeutic agents contained in fluid preparation 406 can be delivered to the subject for systemic uptake.

Upon completing delivery of fluid preparation 406, one or more components of needle 420 (e.g., tissue piercing member 424) can subsequently degrade within the GI lumen wall or surrounding tissue, or other area within the GI tract, along with one or more additional components of the device (e.g., housing 402, membrane 415, piston 419). Further, deflation valve 212 can release a substantial amount of the gas contained within interior 202a to allow for substantial deflation of balloon 202 and subsequent traversal of the device through the remainder of the GI tract to exit the anus of the subject.

Referring to FIG. 6, a modular delivery system 500 (another embodiment of modular delivery system 104) is partially shown coupled to elongated section 210 of balloon 202 according to another example. Modular delivery system 500 is structured like modular delivery system 400 discussed above, where like reference numerals refer to like components between embodiments but are increased by an order of 100 (e.g., release mechanism 408 is equivalent to release mechanism 508). However, in this embodiment, modular delivery system 500 includes a nozzle 502d and a nozzle member 524 structured to deliver the fluid preparation (not shown) as a fluid jet from the device into the GI lumen wall or surrounding tissue, instead of via tissue piercing member 424.

As shown in FIG. 6, modular delivery system 500 includes a housing 502 defining a second chamber 502b with a distal end 502d. Distal end 502d tapers inwardly toward a second axis 502b′ to define a nozzle 502e. Nozzle 502e defines a nozzle opening 502e′ for discharging fluid preparation as a fluid jet stream from the device. In other embodiments, a separate nozzle may be coupled to, or integrally formed with (e.g., insert molded), housing 502. A seal 503 (e.g., a foil) may be coupled at distal end 502d below nozzle opening 502e′ to substantially prevent fluid within the GI tract from entering second chamber 502b.

Modular delivery system 500 further includes a nozzle member 524 coupled at a distal end of needle sleeve 522. Nozzle member 524 defines a discharge opening 524′ for discharging the fluid preparation. Nozzle member 524 is also structured to pierce through seal 503 upon sufficient axial movement of piston 519 relative to housing 502 such that nozzle member 524 is at least partially seated in nozzle opening 502e′. For example, nozzle member 524 is shown to include a protrusion for puncturing seal 503 and seating within nozzle opening 502e′. The engagement between nozzle member 524 and nozzle 502e may provide the necessary resistance to cause seal piercing member 526 to pierce through the reservoir seal (not shown) for discharging the fluid preparation in a similar manner as discussed above with respect to FIG. 5. In this way, the fluid preparation contained in modular delivery system 500 can be expelled through discharge opening 524′ and nozzle opening 502e′ as a fluid jet into the GI lumen wall or surrounding tissue.

Modular delivery system 500 may include suitable amounts of reactants for generating sufficiently high local pressures within housing 502 to discharge the fluid preparation as a fluid jet with enough force to penetrate the GI lumen wall. For example, modular delivery system 500 may include enough reactants to generate local pressures in a range of about 150 psi to about 300 psi within modular delivery system 500 for the fluid jet to penetrate the lumen wall tissue.

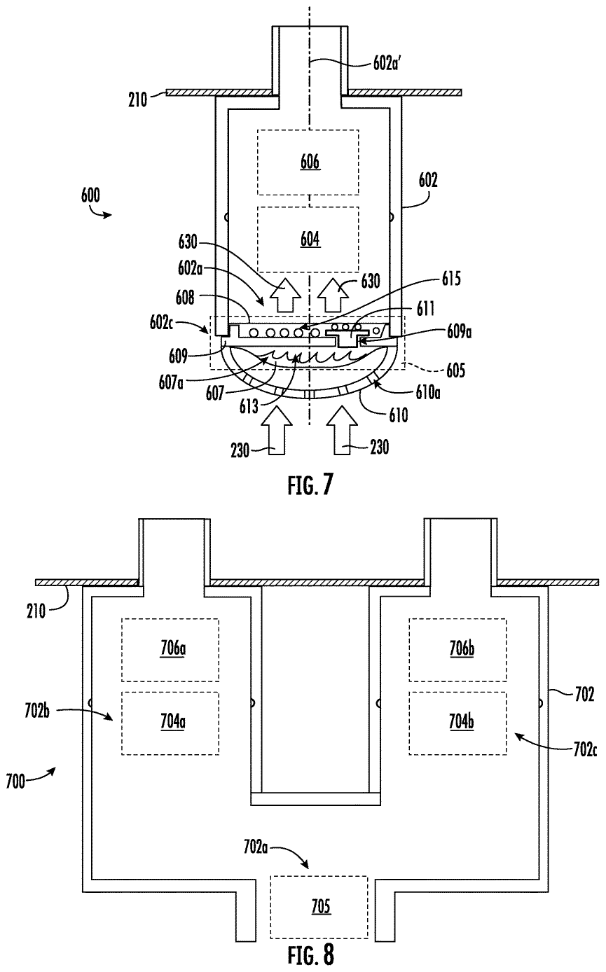

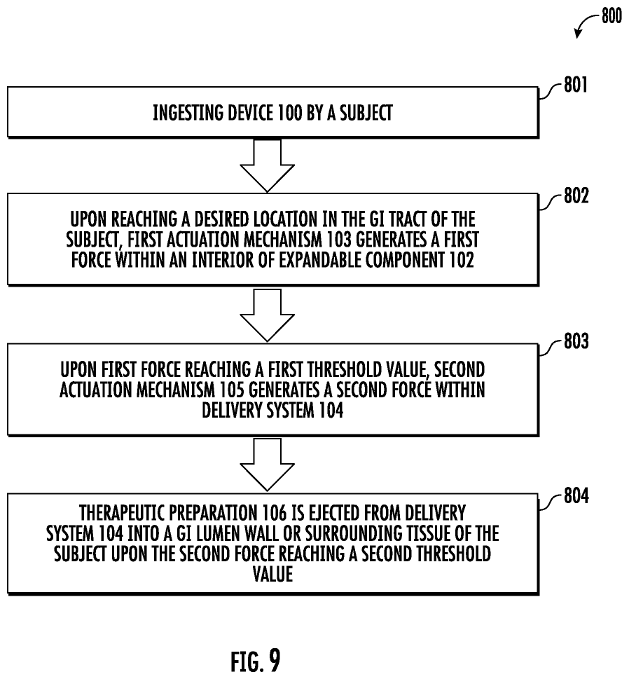

Referring to FIG. 7, a modular delivery system 600 (another embodiment of modular delivery system 104) is shown coupled to elongated section 210 of balloon 202 according to another example. This embodiment illustrates another example of second actuation mechanism 105, which is shown as a second gas generating mechanism 605 in FIG. 7. Modular delivery system 600 is shown to further include a housing 602 (another embodiment of housing 107), a delivery component 604, and a therapeutic preparation 606 (another embodiment of therapeutic preparation 106). Delivery component 604 and therapeutic preparation 606 are shown schematically in FIG. 7, although it should be appreciated that delivery component 604 and therapeutic preparation 606 may have a structure corresponding to any of the examples discussed herein.

As shown in FIG. 7, housing 602 has a generally cylindrical shape defining a first chamber 602a having a central longitudinal axis 602a′. Although not depicted in FIG. 7, housing 602 may further define a second chamber for temporarily storing therapeutic preparation 606. Delivery component 604 may be disposed between first chamber 602a and the second chamber. Delivery component 604 may include a release mechanism (e.g., trigger) coupled to housing 602 within the second chamber or within first chamber 602a adjacent to second gas generating mechanism 605. For example, the release mechanism may be structured similarly as release 408 discussed herein with respect to FIG. 5.

Still referring to FIG. 7, second gas generating mechanism 605 is disposed in first chamber 602a at a proximal end 602c of housing 602. Second gas generating mechanism 605 includes a bladder 607, a substrate 609, a plug 611, a first substance 613, and a second substance 615. Second gas generating mechanism 605 may optionally include a cover 610 and a reactant seal 608. Second gas generating mechanism 605 may define a modular assembly that can be assembled separately from housing 602 and then coupled to housing 602 as a single unit.

Bladder 607 is coupled to substrate 609 to define an interior volume 607a for containing first substance 613. For example, bladder 607 may be in the form of a sheet made from a flexible material (e.g., PET). Bladder 607 may be heat sealed, or otherwise attached, along a periphery of substrate 609 to define interior volume 607a. An optional cover 610 is shown coupled to substrate 609 with bladder 607 disposed therebetween. Cover 610 includes one or more openings 610a for allowing gas from within interior 202a of balloon 202 to reach an outer surface of bladder 607. As discussed below, bladder 607 is structured to be deformed by the gas pressure generated within interior 202a of balloon 202 when the gas pressure is applied against an outer surface of bladder 607. Bladder 607 is further structured to substantially contain the gas pressure generated by second gas generating mechanism 605 within housing 602.

Substrate 609 has a generally planar structure and is coupled (e.g., heat staked, ultrasonically welded, adhered) to housing 602 at proximal end 602c. Substrate 609 further includes an opening 609a for receiving plug 611 therein. Plug 611 is detachably coupled to substrate 609 at opening 609a to temporarily separate first substance 613 from second substance 615. Plug 611 can create a substantially fluid tight seal with substrate 609 for separating first substance 613 from second substance 615. For example, plug 611 may be formed from a flexible polymeric material (e.g., silicone) and may be sized to create an interference fit with substrate 609 when plug 611 is inserted into opening 609a. Plug 611 may include a flange and/or have a tapered shape for maintaining the substantially fluid tight seal between plug 611 and substrate 609. As discussed below, plug 611 is structured to detach from substrate 609 in response to a pressure applied by first substance 613 and bladder 607 when the gas pressure generated within interior 202a reaches or exceeds the first threshold pressure 230.

First substance 613 is disposed within interior volume 607a between bladder 607 and substrate 609. First substance 613 may be in a fluid form (e.g., a liquid, a gel). For example, first substance 613 may be an acid, such as citric acid.

Second substance 615 is disposed in first chamber 602a on an opposite side of substrate 609 separate from first substance 613, although it should be appreciated that the locations of first substance 613 and second substance 615 may be reversed according to other examples. Second substance 615 may be in a powder or a solid form, such as a tablet. For example, second substance 615 may be a carbonate, such as potassium bicarbonate. Second substance 615 is configured to react with first substance 613 to generate a gas (e.g., CO₂) within housing 602. An optional reactant seal 608 (e.g., a foil) may be coupled (e.g., adhered) to substrate 609 to substantially contain second substance 615 within second gas generating mechanism 605 and thereby define a modular actuation assembly. Reactant seal 608 may be structured to tear or otherwise open to release the gas generated upon mixing first substance 613 with second substance 615. In other examples, second substance 615 may be disposed directly within first chamber 602a without reactant seal 608.

The function of delivery system 600 will now be discussed when balloon 202 is inflated at a desired location in a GI tract of a subject for delivery of therapeutic preparation 606. The first gas pressure generated within interior 202a of balloon 202 is applied against an outer surface of bladder 607 through openings 610a of cover 610. Upon the first gas pressure reaching or exceeding the first threshold pressure 230, the first gas pressure causes bladder 607 to deform inwardly toward substrate 609. This inward movement of bladder 607 pushes first substance 613 against substrate 609 to thereby cause plug 611 to detach from substrate 609. Detaching plug 611 from substrate 609 allows first substance 613 to mix with second substance 615 via opening 609a within first chamber 602a to generate a second gas pressure within modular delivery system 600. Upon the second gas pressure reaching or exceeding a second threshold value 630, the second gas pressure can cause reactant seal 608 to open to actuate delivery component 604 and thereby deliver therapeutic preparation 606 from housing 602 into a GI lumen wall or surrounding tissue thereof.

In another example, instead of using a plug 611, delivery system 600 may include a valve (e.g., a one-way valve) that is structured to open in response to the pressure from first substance 613 and bladder 607 to thereby allow first substance 613 to mix with second substance 615 in first chamber 602a.

Referring to FIG. 8, a modular delivery system 700 (another embodiment of modular delivery system 104) is shown coupled to balloon 202 along elongated section 210 according to another example. In this example, modular delivery system 700 is structured to deliver a plurality of therapeutic preparations. More specifically, modular delivery system 700 is structured to deliver a first therapeutic preparation 706a and a second therapeutic preparation 706b. In other examples, modular delivery system 700 may be structured to deliver more than two therapeutic preparations. First therapeutic preparation 706a may be the same as, or different from, second therapeutic preparation 706b, depending on a desired therapeutic effect to be achieved in a subject. For example, modular delivery system 700 may be structured to deliver an incretin combination for the treatment of, for example, a weight disorder, where delivery system 700 includes a first therapeutic preparation 706a having a first therapeutic agent (e.g., one of a GLP-1, GLP-2, GIP, glucagon, or PYY) and a second therapeutic preparation 706b having a second therapeutic agent (e.g., one of a GLP-1, GLP-2, GIP, glucagon, or PYY) that is different from the first therapeutic agent.

As shown in FIG. 8, modular delivery system 700 further includes a housing 702 (another embodiment of housing 107) defining a first chamber 702a, a second chamber 702b, and a third chamber 702c. First chamber 702a, second chamber 702b, and third chamber 702c are in fluid communication with each other. First chamber 702a includes a second gas generating mechanism 705 (another embodiment of second actuation mechanism 105) disposed therein. Second chamber 702b includes a first delivery component 704a and first therapeutic preparation 706a disposed therein. Third chamber 702c includes a second delivery component 704b and second therapeutic preparation 706b disposed therein. Second gas generating mechanism 705, first and second delivery components 704a, 704b, and first and second therapeutic preparation 706a, 706b are shown schematically in FIG. 7. However, it should be appreciated that these components may be structured as any one of the corresponding examples discussed above in FIGS. 1-7.

Modular delivery system 700 is structured such that second gas generating mechanism 705 is actuated in response to the first gas pressure generated within interior 202a of balloon 202 reaching or exceeding first threshold gas pressure 230. Second gas generating mechanism 705 is structured to generate a second gas pressure within housing 702 to simultaneously cause first delivery component 704a to deliver first therapeutic preparation 706a into a GI lumen wall or surrounding tissue thereof and second delivery component 704b to deliver second therapeutic preparation 706b into the GI lumen wall or surrounding tissue thereof. First delivery component 704a and second delivery component 704b may be structured to actuate upon the second gas pressure reaching or exceeding a second threshold gas pressure. In other examples, first delivery component 704a may be structured to actuate at a threshold gas pressure that is different (e.g., lower) than a threshold gas pressure to actuate second delivery component 704b to thereby facilitate sequential delivery of first therapeutic preparation 706a and second therapeutic preparation 706b.

Referring to FIG. 8, a method 800 of delivering therapeutic preparation 106 into a GI lumen wall or surrounding tissue of a subject using ingestible device 100, or use of ingestible device 100 to deliver therapeutic preparation 106 into a GI lumen wall or surrounding tissue of a subject, is shown.

In a first step 801, a subject ingests, or is instructed to ingest, ingestible device 100. For example, the subject may swallow, or may be instructed to swallow, ingestible device 100.

In a second step 802, upon ingestible device 100 reaching a desired location (e.g., stomach, intestine) in the GI tract of the subject for delivery of therapeutic preparation 106, first actuation mechanism 103 generates a first force to cause expandable component 102 to expand. For example, first actuation mechanism 103 may be a first gas generating mechanism that generates a first gas pressure within expandable component 102. Expandable component 102 may be a balloon structured to inflate in response to the first gas pressure.

In a third step 803, upon the first force reaching or exceeding a first threshold value, second actuation mechanism 105 generates a second force within modular delivery system 104. For example, second actuation mechanism 105 may be a second gas generating mechanism that generates a second gas pressure within modular delivery system 104.

In a fourth step 804, upon the second force reaching or exceeding a second threshold value, modular delivery system 104 is structured to deliver therapeutic preparation 106 into the GI lumen wall or surrounding tissue of the subject. For example, modular delivery system 104 may include a delivery component (e.g., a delivery piston) structured to deliver therapeutic preparation 106 from modular delivery system 104 into the GI lumen wall or surrounding tissue in response to the second gas pressure reaching or exceeding the second threshold value. Upon delivery of therapeutic preparation 106 into the GI lumen wall or surrounding tissue, therapeutic preparation 106 can release one or more therapeutic agents contained therein into the subject's body for systemic uptake.

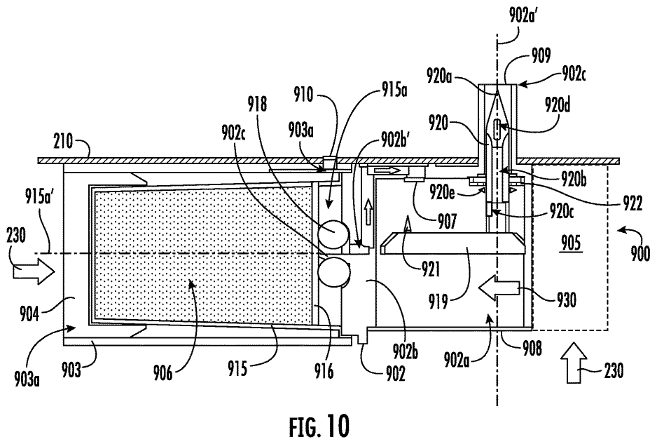

Referring now to FIG. 10, a delivery system 900 (another embodiment of delivery system 104) is shown coupled to elongated section 210 of balloon 202 according to another example. In this embodiment, delivery system 900 is structured to deliver a therapeutic preparation in the form of a fluid preparation 906 (another embodiment of therapeutic preparation 106). Delivery system 900 is shown to include a first housing 902 (another embodiment of housing 107), an optional second actuation mechanism 905 (another embodiment of second actuation mechanism 105), a drug container 915, fluid preparation 906, an optional second housing 903 and second piston 904, a first piston 919, and a needle 920.

In this embodiment, a separate drug container 915 is coupled to a lateral wall of first housing 902 and extends in a lateral direction, whereas first piston 919 and needle 920 are oriented in a longitudinal direction within a piston chamber 902a of first housing 902. Drug container 915 is in selective fluid communication with piston chamber 902a to deliver fluid preparation 906 into a GI lumen wall or surrounding tissue via needle 920. As discussed below, when the gas that pressurizes balloon 202 reaches or exceeds first threshold gas pressure 230, second actuation mechanism 905 (if present) is actuated to generate a second gas pressure within piston chamber 902a of first housing 902. When the second gas pressure reaches or exceeds a second threshold gas pressure (represented by directional arrow 930), the second gas pressure causes piston 919 and needle 920 to move longitudinally within piston chamber 902a to penetrate the GI lumen wall. The second gas pressure is also applied either directly to drug container 915 or indirectly to drug container 915 via optional second piston 904 (if present). As a result, the second gas pressure causes drug container 915 to deform (e.g., collapse) laterally toward first housing 902 to expel fluid preparation 906 from drug container 915 through a drug channel 902c of first housing 902 and into needle 920 for delivery into the GI lumen wall or surrounding tissue thereof.

Still referring to FIG. 10, first housing 902 has a generally hollow cylindrical shape to define piston chamber 902a. Piston chamber 902a extends to a distal end 902c to define a longitudinal axis 902a′. Second actuation mechanism 905 is shown coupled to a lateral wall 902b of first housing 902 and is in fluid communication with piston chamber 902a (e.g., via one or more openings in lateral wall 902b). In other examples, second actuation mechanism 905 may be coupled to a proximal end of first housing 902 adjacent piston chamber 902a. Second actuation mechanism 905 is shown schematically in FIG. 10, but may be structured as any one of, or a combination of, the various embodiments of second actuation mechanism 105 discussed herein. A cover 908 may be coupled to first housing 902 at a proximal end thereof. In other examples, housing 902 may be enclosed at the proximal end without a separate cover 908.

According to another example, delivery system 900 may be structured to function without second actuation mechanism 905. For example, the gas pressure generated in balloon 202 by a first actuation mechanism may be sufficient to actuate first piston 919 through one or more openings disposed in cover 908. The openings in cover 908 may permit the gas pressure generated within balloon interior 202a to enter piston chamber 902a and apply a force against first piston 919.

Still referring to FIG. 10, lateral wall 902b includes a section for interfacing with drug container 915. More specifically, lateral wall 902b includes a fluid channel 902b′ extending from an outermost surface of lateral wall 902b into piston chamber 902a. A drug port seal 907 is coupled to first housing 902 to temporarily block fluid flow from fluid channel 902b′ to piston chamber 902a. As discussed herein, drug port seal 907 is structured to open in response to sufficient axial movement of first piston 919 relative to first housing 902. Drug port seal 907 may be structured as a film, a foil, a detachable plug, or other structure for temporarily preventing fluid flow through fluid channel 902b′ into piston chamber 902a.

Lateral wall 902b further includes a drug container seal piercer 902c extending outwardly therefrom. Drug container seal piercer 902c is located adjacent to fluid channel 902b'. Drug container seal piercer 902c extends to a distal tip positioned adjacent to a drug container seal 916 of drug container 915. The distal tip of drug container seal piercer 902c is structured to pierce drug container seal 916 to allow fluid preparation 906 to flow through fluid channel 902b′ and into piston chamber 902a. A channel seal 918 is coupled to first housing 902 between drug container seal piercer 902c and drug seal 916. Channel seal 918 is structured to direct fluid preparation 906 from drug container 915 into fluid channel 902b′. In the example shown, channel seal 918 is structured as an O-ring. However, in other examples, channel seal 918 may be structured differently, such as a gasket or other sealing structure.

Drug container 915 is coupled (e.g., heat sealed, adhered) to lateral wall 902b in a lateral orientation with first piston 919 and needle 920 oriented asymmetrically relative to drug container 915. In this manner, delivery system 900 may help to optimize the amount of drug contained in the device while minimizing the delivery system package size within balloon interior 202a. Further, the lateral orientation of drug container 915 may help to facilitate consistent orientation (e.g., upon unfolding of balloon 202 within a GI lumen) of first piston 919 and needle 920 relative to a GI lumen wall for delivery of fluid preparation 906.

Drug container 915 has a generally hollow cylindrical shape to define an interior 915a and a lateral axis 915a′. Interior 915a may define a volume for containing up to about 400 μl of fluid, including about 50 μl, 100 μl, 200 μl, 300 μl, 400 μl, or any value therebetween. In other examples, interior 915a may define a volume for containing about 200-300 μl of fluid, including about 200 μl, 250 μl, 300 μl, or any value therebetween. Drug container 915 is structured to be deformed (e.g., compressed, constricted) in a lateral direction along lateral axis 915a′ toward first housing 902 by the gas pressure generated within balloon interior 202a to thereby expel fluid preparation 906. For example, drug container 915 may be formed from, or include, a deformable material, such as PET, or other flexible material or combinations of materials. Drug container 915 may further have a tapered (e.g., frusto-conical) profile to help facilitate lateral deformation in response to the generated gas pressure. Drug container seal 916 is coupled to an end of drug container 915 located adjacent to drug container seal piercer 902c. Drug container seal 916 may be a film, a foil, or other type of seal that is structured to be punctured or otherwise opened by drug container seal piercer 902c.

Drug container 915 and/or drug container seal 916 may comprise a metalized film or coating (e.g., titanium film) to help minimize water vapor transmission into the interior of drug container 915 and thereby help to prolong the shelf-life of fluid preparation 906. The metalized film may be applied to an outer exposed surface of drug container 915 and/or drug container seal 916.

Delivery system 900 may optionally include second housing 903 and second piston 904 to, for example, provide an independent vent path from first housing 902 and first piston 919 to vent pressurized gas following deformation of drug container 915. As shown in FIG. 10, second housing 903 is coupled to first housing 902 at lateral wall 902b and partially surrounds drug container 915. Second housing 903 includes an open end 903a for slidably receiving second piston 904 therein. Second housing 903 further includes a vent channel 903b extending through a side wall of second housing 903 to ambient (e.g., to a GI lumen environment external to delivery system 900 and balloon 202) for venting pressurized gas from within second housing 903 after axial movement of second piston 904 and subsequent deformation of drug container 915.

A degradable plug 910 may be coupled to second housing 903 to temporarily block vent channel 903b from the GI lumen environment to, for example, help to prevent ingress of GI fluid. Degradable plug 910 may comprise a degradable material, such as an enteric material.

As discussed below, second piston 904 is structured to move (e.g., slide) along lateral axis 915a′ in response to the generated gas pressure in balloon interior 202a to thereby deform drug container 915 to expel fluid preparation 906. Vent channel 903b is in selective fluid communication with the GI lumen environment to allow pressurized air generated within second housing 903 to be discharged from the device after second piston 904 moves past vent channel 903b when second piston 904 has expelled a substantial amount of fluid preparation 906. Second housing 903 and/or second piston 904 may be formed from, or include, a biodegradable material, such as PEO, PVA, or other degradable material or combinations of materials to allow for at least partial biodegradation within the GI tract of a subject.

Still referring to FIG. 10, first piston 919 is slidably disposed in piston chamber 902a. First piston 919 further includes a drug port seal piercer 921 coupled to, or integrally formed with, an upper surface of first piston 919. Drug port seal piercer 921 is structured to pierce drug port seal 907 upon sufficient longitudinal movement of first piston 919 relative to first housing 902 to allow fluid preparation 906 to flow from fluid channel 902b′ to needle 920. First piston 919 may be formed from a polymeric material (e.g., ABS), or other material or combinations of materials.

Needle 920 is coupled to first piston 919. Needle 920 extends longitudinally from first piston 919 to a distal tip 920a along longitudinal axis 902a′. Needle 920 is structured to penetrate a GI lumen wall or surrounding tissue and to deliver fluid preparation 906 thereto. Needle 920 further includes an inner channel 920b extending from an inlet 920c to an outlet 920d. Inlet 920c is disposed in a side wall of needle 920 to allow fluid preparation 906 to flow into inner channel 920b upon drug port seal piercer 921 piercing drug port seal 907. In various embodiments, needle 920 may be structured similarly as tissue piercing member 424 discussed herein.

Needle seal 909 is coupled (e.g., adhered) to first housing 902 at a distal end 902c of first housing 902 to substantially prevent fluid in the GI tract from entering into piston chamber 902a until needle 920 pierces needle seal 909. Needle seal 909 may be formed from a penetrable material (e.g., aluminum foil) to allow needle 920 to pierce through needle seal 909. In this way, needle seal 909 and first housing 902 can cooperatively define a substantially sterile environment for containing needle 920 before delivery of fluid preparation 906 into the GI lumen wall or surrounding tissue.

A release mechanism 922 is coupled to (e.g., press-fit, snap-fit, adhered), or integrally formed with, first housing 902 within piston chamber 902a. As shown in FIG. 10, release mechanism 922 is coupled to an inner surface of first housing 902 within piston chamber 902a. Release mechanism 922 is structured to interface with first piston 919 and needle 920 to substantially prevent advancement of first piston 919 and needle 920 until sufficient pressure is generated by the second gas within piston chamber 902a. For example, release mechanism 922 may include one or more fingers extending radially inward toward longitudinal axis 919a. Each of the fingers may extend radially inward in a cantilevered manner from a circumferential edge of release mechanism 922 to a free end located adjacent needle 920. The fingers may be structured to interface with a protrusion 920e of needle 920 via an interference (e.g., overlapping) condition to substantially prevent needle 920 from piercing needle seal 909 until sufficient pressure is generated within piston chamber 902a. When the second gas pressure reaches or exceeds second threshold pressure 930, protrusion 920e overcomes the interference condition with the fingers of release mechanism 922 by causing the fingers to deflect away from needle 920 to thereby allow first piston 919 and needle 920 to move axially along longitudinal axis 919a toward needle seal 909, such that needle 920 pierces needle seal 909.

In other examples, release mechanism 922 may be structured as any one of, or a combination of, the other release mechanisms discussed herein.

The function of delivery system 900 will now be discussed when balloon 202 is inflated at a desired location of a GI tract of a subject for delivery of fluid preparation 906. The generated gas within interior 202a of balloon 202 applies a first gas pressure against second gas generating mechanism 905. Upon the first gas pressure reaching or exceeding first threshold pressure 230 (e.g., a pressure associated with a partially or fully inflated state of balloon 202), second gas generating mechanism 905 (if present) is actuated to generate a second gas pressure within piston chamber 902a. Alternatively, in embodiments without second gas generating mechanism 905, the first threshold pressure 230 generated within interior 202a may be sufficient to actuate first piston 919.

In the embodiment shown in FIG. 10, when the second gas pressure reaches or exceeds second threshold pressure 930, protrusion 920e overcomes the interference condition with release mechanism 922. As a result, piston 919 moves axially relative to first housing 902 along longitudinal axis 902a′ toward the GI lumen wall such that distal tip 920a of needle 920 is advanced through needle seal 909 to penetrate the GI lumen wall.

The generated gas pressure within balloon interior 202a is also applied against drug container 915, either directly against container 915 or indirectly via optional second piston 904 (if present). Upon the generated gas pressure reaching or exceeding a threshold drug delivery pressure value, drug container 915 is deformed (e.g., compressed) by the generated gas pressure to cause drug container seal piercer 902c to pierce drug container seal 916. The threshold drug delivery pressure value may be selected such that fluid preparation 906 is released from drug container 915 before, substantially simultaneously with, or after drug port seal piercer 921 pierces drug port seal 907. In some embodiments, the threshold drug delivery pressure value may be substantially the same as first threshold pressure 230. In other embodiments, the threshold drug delivery pressure value may be substantially the same as second threshold pressure 930. Upon piercing drug container seal 916, the generated gas pressure in balloon interior 202a causes drug container 915 to sufficiently deform to evacuate fluid preparation 906 from drug container 915 into fluid channel 902b′.

Upon sufficient axial movement of first piston 919 relative to first housing 902, drug port seal piercer 921 pierces drug port seal 907 to allow fluid preparation 906 to flow from fluid channel 902b′ into piston chamber 902a between first piston 919 and first housing 902. Fluid preparation 906 is then directed into inner channel 920b of needle 920 via inlet 920c where fluid preparation 906 is directed through outlet 920d into a GI lumen wall or surrounding tissue for systemic uptake of one or more therapeutic agents contained in fluid preparation 906.

The foregoing description of various embodiments has been presented for purposes of illustration and description. It is not intended to limit the invention to the precise forms disclosed. Many modifications, variations and refinements will be apparent to practitioners skilled in the art. For example, embodiments of the device can be sized and otherwise adapted for various pediatric and neonatal applications as well as various veterinary applications. Also, those skilled in the art will recognize, or be able to ascertain using no more than routine experimentation, numerous equivalents to the specific devices and methods described herein. Such equivalents are considered to be within the scope of the present disclosure.

While the present disclosure has been described and illustrated with reference to specific embodiments thereof, these descriptions and illustrations do not limit the present disclosure. It can be clearly understood that various changes can be made, and equivalent components can be substituted within the embodiments, without departing from the true spirit and scope of the present disclosure. Also, components, characteristics, or acts from one embodiment can be readily recombined or substituted with one or more components, characteristics or acts from other embodiments to form numerous additional embodiments within the scope of the invention. Moreover, components that are shown or described as being combined with other components, can, in various embodiments, exist as standalone components. Further, for any positive recitation of a component, characteristic, constituent, feature, step or the like, embodiments of the invention specifically contemplate the exclusion of that component, value, characteristic, constituent, feature, step or the like. The illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. The specification and drawings are to be regarded as illustrative rather than restrictive. Modifications can be made to adapt a particular situation, material, composition of matter, method, or process to the objective, spirit and scope of the present disclosure. All such modifications are intended to be within the scope of the present disclosure. While the methods disclosed herein have been described with reference to particular operations performed in a particular order, it can be understood that these operations can be combined, sub-divided, or re-ordered to form an equivalent method without departing from the teachings of the present disclosure. Therefore, unless specifically indicated herein, the order and grouping of the operations are not limitations of the present disclosure.

What is claimed is:

1. An ingestible device for delivering a therapeutic preparation into a GI lumen wall or surrounding tissue of a subject, the device comprising:
an expandable component comprising a first actuation mechanism; and
a delivery system coupled to the expandable component, the delivery system comprising a second actuation mechanism and the therapeutic preparation, the therapeutic preparation comprising at least one therapeutic agent;
wherein the first actuation mechanism is structured to generate a first force in response to a condition in the GI tract of the subject to cause the expandable component to expand so as to position the delivery system relative to a surface of the GI lumen wall;
wherein the second actuation mechanism is structured to generate a second force within the delivery system in response to the first force reaching a first threshold value; and
wherein the delivery system is structured to deliver the therapeutic preparation into the GI lumen wall or surrounding tissue upon the second force reaching a second threshold value.

2. The device of claim 1, wherein the first threshold value is associated with an expanded state of the expandable component within the GI tract.

3. The device of claim 1, wherein the second threshold value is greater than the first threshold value.

4. The device of any one of claims 1-3, wherein the first actuation mechanism comprises a first gas generating mechanism structured to generate a first gas pressure to provide the first force.

5. The device of claim 4, wherein the first gas generating mechanism comprises a first plurality of substances that when mixed undergo a first chemical reaction to generate the first gas pressure.

6. The device of claim 5, wherein the first chemical reaction is initiated in response to the condition in the GI tract of the subject.

7. The device of claim 6, wherein the condition is a pH in the small intestine.

8. The device of any one of claims 4-7, wherein the first gas pressure is in a range of about 5-20 psi.

9. The device of any one of the preceding claims, wherein the second actuation mechanism comprises a second gas generating mechanism structured to generate a second gas pressure to provide the second force.

10. The device of claim 9, wherein the second gas generating mechanism comprises a second plurality of substances that when mixed undergo a second chemical reaction to generate the second gas pressure.

11. The device of claim 9 or 10, wherein the second gas pressure is in a range of about 25-250 psi.

12. The device of any one of the preceding claims, wherein the second threshold value corresponds with a desired penetration depth in the subject for delivery of the therapeutic preparation.

13. The device of claim 12, wherein the desired penetration depth is into a peritoneum or a peritoneal cavity of the subject.

14. The device of claim 12, wherein the desired penetration depth is into a submucosa of the GI lumen wall.

15. The device of any one of the preceding claims, wherein the second force is substantially contained within an interior of the delivery system.

16. The device of any one of the preceding claims, wherein the first force is substantially contained within the expandable component external to the delivery system.

17. The device of any one of the preceding claims, wherein the therapeutic preparation is a first therapeutic preparation and the delivery system further comprises a second therapeutic preparation.

18. The device of claim 17, wherein the first therapeutic preparation includes the at least one therapeutic agent, and the second therapeutic preparation includes a second therapeutic agent.

19. The device of claim 18, wherein the at least one therapeutic agent is the same as, or different from, the second therapeutic agent.

20. The device of claim 1, wherein the delivery system is a first delivery system and the therapeutic preparation is a first therapeutic preparation, wherein the device further comprises a second delivery system coupled to the expandable component, and wherein the second delivery system comprises a second therapeutic preparation.

21. The device of claim 1, wherein the delivery system comprises a housing and a delivery component, wherein the housing includes a first chamber and a second chamber, and wherein the delivery component is movably disposed in the housing.

22. The device of claim 21, wherein the first chamber is oriented perpendicularly relative to the second chamber.

23. The device of claim 21 or 22, wherein the second actuation mechanism is disposed in the first chamber, and wherein the therapeutic preparation is disposed in the second chamber.

24. The device of any one of claims 21-23, wherein the second actuation mechanism comprises a first substance, a second substance, and a separator, wherein the first and second substances are disposed in the first chamber and are separated from each other by the separator.

25. The device of claim 24, wherein the device is structured such that the first force causes the first and second substances to mix to generate a gas pressure to provide the second force within the first chamber, and wherein the delivery component is structured to deliver the therapeutic preparation from the second chamber in response to the generated gas pressure.

26. The device of any one of claims 24-25, wherein the separator comprises a membrane.

27. The device of claim 26, wherein the membrane is structured to open in response to a pressure applied to the membrane.

28. The device of claim 26 or 27, wherein the membrane is a pierceable membrane.

29. The device of claim 28, wherein the delivery system further comprises an actuation piston structured to pierce the pierceable membrane in response to the first force to cause the first and second substances to mix.

30. The device of any one of claims 24-25, wherein the separator comprises a substrate with an opening disposed therein and a plug detachably coupled to the substrate at the opening.

31. The device of claim 30, wherein the delivery system further comprises a compressible bladder structured to compress in response to the first force to detach the plug from the substrate to cause the first and second substances to mix.

32. The device of any one of claims 30-31, wherein the delivery system further comprises a valve structured to open in response to the first force to cause the first and second substances to mix.

33. The device of any one of claims 21-32, wherein the therapeutic preparation is delivered in a fluid form from the second chamber.

34. The device of claim 33, wherein the housing includes a nozzle to deliver the therapeutic preparation as a fluid jet from the second chamber into the GI lumen wall or surrounding tissue thereof.

35. The device of claim 33, wherein the delivery system further comprises a hollow needle coupled to the delivery component, and wherein the needle is structured to penetrate the GI lumen wall or surrounding tissue to deliver the fluid form of the therapeutic preparation.

36. The device of any one of claims 21-32, wherein the therapeutic preparation comprises a biodegradable needle including a cavity and a solid tablet comprising the therapeutic agent, and wherein the solid tablet is disposed in the cavity.

37. The device of any one of claims 21-32, wherein the therapeutic preparation is formed into the shape of a needle.

38. The device of any one of the preceding claims, wherein the expandable component comprises a balloon.

39. The device of claim 38, wherein the balloon comprises a biodegradable material such that at least a portion of the balloon degrades within the GI tract of the subject upon delivery of the therapeutic preparation.

40. A method of delivering a therapeutic preparation into a GI lumen wall or surrounding tissue of a subject, the method comprising orally administering to the subject by swallowing an ingestible device of any one of claims 1-39.

41. A delivery system for an ingestible device, the delivery system comprising:
a first housing having a piston chamber, the piston chamber defining a longitudinal axis;
a first piston disposed in the piston chamber;
a needle coupled to the first piston;
a drug container coupled to the first housing and in selective fluid communication with the piston chamber, the drug container oriented laterally relative to the first housing; and
a fluid preparation disposed in the drug container;
wherein the drug container comprises a flexible material structured to deform upon ingestion of the device to expel the fluid preparation into the piston chamber.

42. The delivery system of claim 41, further comprising:
a second housing surrounding at least a portion of the drug container; and
a second piston disposed in the second housing adjacent to the drug container.

43. The delivery system of claim 42, wherein the first piston is structured to move in a longitudinal direction within the first housing to cause the needle to penetrate a GI lumen wall, and wherein the second piston is structured to move in a lateral direction within the second housing to deform the drug container to expel the fluid preparation.

44. The delivery system of claim 42, wherein the second housing comprises a vent channel to vent gas to a GI lumen environment.

45. An ingestible device comprising:
an expandable component comprising a first actuation mechanism; and
the delivery system of any one of claims 41-44 coupled to the expandable component.

46. An ingestible device for delivering a therapeutic preparation into a GI lumen wall or surrounding tissue of a subject, the device comprising:
an expandable component;
a modular delivery system coupled to the expandable component; and
a therapeutic preparation disposed in the modular delivery system, the therapeutic preparation comprising at least one therapeutic agent;
wherein the expandable component comprises a first gas generating mechanism structured to generate a first gas pressure within the expandable component to cause the expandable component to expand in a GI lumen to position the modular delivery system relative to a surface of the GI lumen wall; and
wherein the modular delivery system comprises a second gas generating mechanism structured to generate a second gas pressure within the modular delivery system in response to the first gas pressure to deliver the therapeutic preparation into the GI lumen wall or surrounding tissue.

47. An ingestible device for delivering a therapeutic preparation into a GI lumen wall or surrounding tissue of a subject, the device comprising:
an expandable component;
a modular delivery system coupled to the expandable component; and
a therapeutic preparation disposed in the modular delivery system, the therapeutic preparation comprising at least one therapeutic agent;
wherein the expandable component is structured to expand in response to a condition in the GI tract of the subject to position the delivery system relative to a surface of the GI lumen wall;
wherein the expandable component comprises a first actuation mechanism structured to generate a first force within the expandable component;
wherein the modular delivery system comprises a second actuation mechanism structured to generate a second force within the modular delivery system upon the first force reaching a first threshold value; and
wherein the modular delivery system is structured to deliver the therapeutic preparation into the GI lumen wall or surrounding tissue upon the second force reaching a second threshold value.

48. A modular delivery system for use in an ingestible device to deliver a therapeutic preparation into a GI lumen wall or surrounding tissue of a subject, the modular delivery system comprising:
a housing defining a first chamber and a second chamber;
a gas generating mechanism coupled to the housing in the first chamber, the gas generating mechanism being structured to generate a gas pressure within the first chamber in response to a force applied to the modular delivery system;
a therapeutic preparation disposed in the second chamber, the therapeutic preparation comprising at least one therapeutic agent; and
a delivery component movably disposed in the housing between the first chamber and the second chamber, the delivery component being structured to eject the therapeutic preparation from the second chamber in response to the gas pressure generated within the first chamber.

49. A modular delivery system for use in an ingestible device to deliver a therapeutic preparation into a GI lumen wall or surrounding tissue of a subject, the modular delivery system comprising:
a housing defining a first chamber and a second chamber;
a first substance disposed in the first chamber;
a second substance disposed in the first chamber;
a separator coupled to the housing in the first chamber, the separator separating the first substance from the second substance;
a delivery piston movably disposed in the housing; and
a therapeutic preparation disposed in the second chamber, the therapeutic preparation comprising at least one therapeutic agent;
wherein the modular delivery system is structured such that the first and second substances mix to form a gas within the first chamber in response to a force applied to the modular delivery system;
wherein the gas creates a pressure within the first chamber to cause the delivery piston to eject the therapeutic preparation from the second chamber into the GI lumen wall or surrounding tissue.

50. A method for delivering a therapeutic preparation into a GI lumen wall or surrounding tissue of a subject, the method comprising:
ingesting, by the subject, an ingestible device, the device comprising an expandable component, a modular delivery system coupled to the expandable component, and a therapeutic preparation comprising at least one therapeutic agent disposed in the modular delivery system, the expandable component including a first gas generating mechanism and the modular delivery system including a second gas generating mechanism;
wherein when the device reaches a desired location in the GI tract of the subject, the first gas generating mechanism generates a first gas pressure within an interior of the expandable component to cause the expandable component to expand to position the modular delivery system relative to a surface of the GI lumen wall; and
wherein the second gas generating mechanism generates a second gas pressure within the modular delivery system in response to the first gas pressure to deliver the therapeutic preparation into the GI lumen wall or surrounding tissue.

51. A method for delivering a therapeutic preparation into a GI lumen wall or surrounding tissue of a subject, the method comprising:
ingesting, by the subject, an ingestible device, the device comprising an expandable component, a modular delivery system coupled to the expandable component, and a therapeutic preparation comprising at least one therapeutic agent disposed in the modular delivery system, the expandable component including a first actuation mechanism and the modular delivery system including a second actuation mechanism;
wherein when the device reaches a desired location in the GI tract of the subject, the first actuation mechanism generates a first force within an interior of the expandable component;
wherein when the first force reaches a first threshold value, the second actuation mechanism generates a second force within the modular delivery system;
wherein when the second force reaches a second threshold value, the device is structured to deliver the therapeutic preparation into the GI lumen wall or surrounding tissue.
